(19) United States
(12) Patent Application Publication
(10) Pub. No.: US 2024/0041792 A1
(43) Pub. Date: Feb. 8, 2024

(54) TOPICAL COMPOSITIONS AND USES THEREOF

(71) Applicant: TRUETIVA, Inc., Grand Rapids, MI (US)

(72) Inventor: J. Phillip KENNEDY, Grand Rapids, MI (US)

(73) Assignee: TRUETIVA, Inc., Grand Rapids, MI (US)

(21) Appl. No.: 18/359,199

(22) Filed: Jul. 26, 2023

Related U.S. Application Data
(60) Provisional application No. 63/392,309, filed on Jul. 26, 2022.

(51) Int. Cl. A61K 31/047; A61K 9/00; A61K 47/36; A61K 8/73; A61K 45/06; A61Q 19/08; A61K 8/34; A61K 31/375; A61K 8/67; A61K 31/455

(52) U.S. Cl. CPC A61K 31/047; A61K 9/0014; A61K 47/36; A61K 8/735; A61K 45/06; A61Q 19/08; A61K 8/345; A61K 31/375; A61K 8/676; A61K 31/455; A61K 8/675

(57) ABSTRACT

The present invention relates to a topical composition comprising a cannabinoid, extracellular matrix component or extracellular matrix fragment, and a pharmaceutically acceptable carrier, excipient, diluent, reagent, or combinations thereof, and use thereof in the treatment skin disorders, muscle pain, and arthritic pain.

### RELATED APPLICATIONS

This application claims priority from U.S. Provisional Patent Application No. 63/392,309, filed Jul. 26, 2022, the entire contents of which are incorporated herein by reference.

### TECHNICAL FIELD

New topical compositions and methods of treatment are described herein. Exemplary topical compositions containing cannabinoids, cannabidiols, cannabidiol isomers, or cannabidiol analogs, and combinations thereof, may be used for treating skin disorders, muscle pain, or arthritic pain by topically administering the compositions to the skin of a patient in need of treatment.

### BACKGROUND

Skin disorders, including dermatological diseases, affect millions of people worldwide. Indeed, it has been estimated that dermatological diseases such as psoriasis, acne, and the like contribute nearly 1.8% to the global burden of disease measured in disability-adjusted life years (DALYs). In the US alone, acne affects around 50 million people annually.

Many skin disorders, such as psoriasis, have no cure and ongoing treatment is required to manage these disorders. Corticosteroids are typically the first line of treatment for skin disorders such as psoriasis, long-term corticosteroid use can cause thinning of the skin and topical corticosteroids can decrease in effectiveness over time. Treatment of skin disorders such as psoriasis and acne may also involve the use of retinoids, which can increase the skin's sensitivity to the sun, antibiotics, which can lead to resistance, or salicylic acid, which can cause skin irritation.

Accordingly, there is an ongoing need for improved or alternative compositions for treating skin disorders.

### SUMMARY

In one aspect, the present invention provides a topical composition comprising a cannabinoid, an extracellular matrix component or an extracellular matrix fragment, and a pharmaceutically acceptable carrier, excipient, diluent, reagent, or combinations thereof.

In another aspect, the present invention provides a method of treating a skin disorder, muscle pain, or arthritic pain, comprising topically administering to a patient in need of treatment a composition comprising a cannabinoid, an extracellular matrix component or an extracellular matrix fragment, and a pharmaceutically acceptable carrier, excipient, diluent, reagent, or combinations thereof.

In another aspect, the present invention provides use of a cannabinoid, an extracellular matrix component or an extracellular matrix fragment, and a pharmaceutically acceptable carrier, excipient, diluent, reagent, or combinations thereof, in the manufacture of a medicament for treating a skin disorder, muscle pain, or arthritic pain by topical administration.

In yet another aspect, the present invention provides a composition comprising a cannabinoid, an extracellular matrix component or an extracellular matrix fragment, and a pharmaceutically acceptable carrier, excipient, diluent, reagent, or combinations thereoftreating a skin disorder, muscle pain, or arthritic pain by topical administration.

### BRIEF DESCRIPTION OF THE DRAWINGS

Embodiments of the invention will now be described with reference to the following FIGURES, which are intended to be exemplary only, and in which:

FIG. 1 is a comparison of the structure of a PD-L1 dimer bound by BMS-202 (left panel) and cannabidiol (right panel).

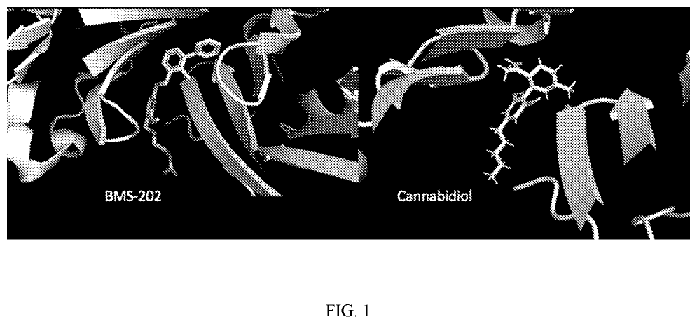

### DETAILED DESCRIPTION

The following description of exemplary topical compositions and methods of treatment should not be used to limit the scope of the present invention. While the invention will be described in conjunction with these specific examples, it will be understood that it is not intended to limit the invention to such specific examples. On the contrary, it is intended to cover alternatives, modifications, and equivalents as may be included within the spirit and scope of the invention. In the following description, numerous specific details are set forth in order to provide a thorough understanding of the present invention. The present invention may be practiced without some or all these specific details. In other instances, well known process operations have not been described in detail so as to unnecessarily obscure the present invention.

Various aspects now will be described more fully hereinafter. Such aspects may, however, be embodied in many different forms and should not be construed as limited to the examples set forth herein; rather, these examples are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 μm, and 7 μm are also intended to be explicitly disclosed, as well as the range of values greater than or equal to 1 μm and the range of values less than or equal to 8 μm.

All percentages, parts and ratios are based upon the total weight of the compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of values that fall within 10%, 9%, 8%, 7%, 6%, 5%, 4%, 3%, 2%, 1%, or less in either direction (greater than or less than) of the stated value unless otherwise stated or otherwise evident from the context (except where such number would exceed 100% of a possible value). In a particular embodiment, the term “about” means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein mean introducing the pharmaceutical composition into the system of the subject in need of treatment, and refer to directly administering a compound (also referred to as an agent of interest) or pharmaceutically acceptable salt of the compound (agent of interest) or a composition to a subject.

When the pharmaceutical composition is provided in combination with one or more other active agents, “administration” and its variants are each understood to include concurrent and/or sequential introduction of the pharmaceutical composition and the other active agents.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In examples or claims where the term comprising is used as the transition phrase, such examples can also be envisioned with replacement of the term “comprising” with the terms “consisting of” or “consisting essentially of.”

As used herein, the term “decoy molecule” refers to any extracellular component or fragment of the cellular surface that binds to intact skin cells, causing plasticity of the extracellular matrix to facilitate transdermal drug delivery.

The term “disorder” is used in this disclosure to mean, and is used interchangeably with the terms disease, condition, syndrome, or illness, unless otherwise indicated.

The terms “effective amount” and “therapeutically effective amount” are used interchangeably in this disclosure and refer to an amount of a compound or composition that, when administered to a subject, is sufficient to treat or reduce a symptom of a disease in a subject or enhance the texture, appearance, color, sensation, or hydration of the intended tissue treatment area. The therapeutically effective amount to be administered will be governed by such considerations, and may be either an incremental maximum tolerated dose or the minimum amount necessary to ameliorate, cure, or treat the condition or one or more of its symptoms. The actual amount which comprises the “effective amount” or “therapeutically effective amount” may also vary depending on a number of conditions including, but not limited to, the severity of the disorder, the size and health of the patient, and the route of administration. In some embodiments, a treatment effective amount is a therapeutically effective amount or a prophylactically effective amount. The term “prophylactically effective amount” refers to an amount effective in preventing or substantially lessening the chances of acquiring a disease or in reducing the severity of the disease before it is acquired or reducing the severity of one or more of its symptoms before the symptoms develop. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc., which are within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g., animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts may be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid. The preparation of the pharmaceutically acceptable salts described above and other typical pharmaceutically acceptable salts is more fully described by Berge et al., “Pharmaceutical Salts,” J. Pharm. Sci., 1977:66:1-19.

The term “patient” and “subject” are interchangeable and may be taken to mean any living organism which may be treated with compounds of the present invention. As such, the terms “patient” and “subject” may include, but is not limited to, any non-human mammal, primate or human. In some examples, the “patient” or “subject” is a mammal, such as mice, rats, other rodents, rabbits, dogs, cats, swine, cattle, sheep, horses, primates, or humans. In some examples, the patient or subject is an adult, child or infant. In some examples, the patient or subject is a human.

The term “skin disorder” as used herein is intended to encompass dermatological diseases, as well as features of the skin that may be considered undesirable, such as changes in desired skin tone (e.g., darkening or hyperpigmentation of the skin, redness of the skin), appearance, texture and/or structure, fine lines and/or wrinkles, reduced skin elasticity and/or sagging, reduced collagen production, signs of aging, dry skin, skin roughness, and the like. Thus, the treating a skin disorder as described herein may comprise treating a dermatological disease, improving (e.g., whitening or lightening) skin tone, appearance, texture and/or structure, reducing fine lines and/or wrinkles, firming and/or lifting of the skin, promoting collagen production, reducing signs of aging, moisturizing skin, reducing skin roughness, and the like.

The terms “treat,” “treating,” or “treatment” are used herein, for instance, in reference to methods of treating a skin disorder, muscle pain, or arthritic pain, or a systemic condition, and generally includes the administration of a compound or composition which reduces the frequency of, or delays the onset of, symptoms of the skin disorder, muscle pain, or arthritic pain or which enhances the texture, appearance, color, sensation, or hydration of the intended tissue treatment area of the tissue surface in a subject relative to a subject not receiving the compound or composition. This may include reversing, reducing, or arresting the symptoms, clinical signs, and underlying pathology of a condition in a manner to improve or stabilize a subject's condition. The terms “treat” “treating” or “treatment” are used herein may also encompass the prevention or prophylaxis of a skin disorder, muscle pain, or arthritic pain, or of an undesirable change in texture, appearance, color, sensation, or hydration of the intended tissue treatment area. The terms “prevention” and “prophylaxis” (and grammatical variations thereof) as used herein refer to administering a medicament in order to avert or forestall the appearance of one or more symptoms of a condition (i.e., a pulmonary disease). The person of ordinary skill in the medical art recognizes that the term “prevent” is not an absolute term. In the medical art, it is understood to refer to the prophylactic administration of a drug to substantially diminish the likelihood or seriousness of a condition, or symptom of the condition and this is the sense intended in this disclosure. As used in a standard text in the field, the Physician's Desk Reference, the terms “prevent”, “preventing,” and “prevention” with regard to a condition refer to averting the cause, effects, symptoms or progression of a condition prior to the condition fully manifesting itself.

As used herein, the term “alkyl” or “alkyl group” means a linear or branched, saturated hydrocarbon including one or more carbon atoms (e.g., one, two, three, four, five, six, seven, eight, nine, ten, or more carbon atoms), which is optionally substituted. The notation “C₁-C₁₀ alkyl” means an optionally substituted linear or branched, saturated hydrocarbon including 1-10 carbon atoms, “C₁-C₆ alkyl” means an optionally substituted linear or branched, saturated hydrocarbon including 1-6 carbon atoms, “C₁-C₃ alkyl” means an optionally substituted linear or branched, saturated hydrocarbon including 1-3 carbon atoms, and the like. Unless otherwise specified, an alkyl group described herein refers to both unsubstituted and substituted alkyl groups. Examples of suitable alkyl groups may include, but are not limited to, methyl, ethyl, 1-propyl, isopropyl, 1-butyl, 2-butyl, isobutyl, sec-butyl, tert-butyl, pentyl, hexyl, heptyl, octyl, nonyl, decyl, and the like.

As used herein, the term “alkenyl” or “alkenyl group” means a linear or branched hydrocarbon including two or more carbon atoms (e.g., two, three, four, five, six, seven, eight, nine, = or more carbon atoms) and at least one double bond, which is optionally substituted. Unless indicated otherwise, the stereochemistry about each double bond may be independently cis or trans, or E or Z, as appropriate. The notation “C₂-C₁₀ alkenyl” means an optionally substituted linear or branched hydrocarbon including 2-10 carbon atoms and at least one carbon-carbon double bond, “C₂-C₆ alkenyl” means an optionally substituted linear or branched hydrocarbon including 2-6 carbon atoms and at least one carbon-carbon double bond, “C₂-C₃ alkenyl” means an optionally substituted linear or branched hydrocarbon including 2-3 carbon atoms and at least one carbon-carbon double bond, and the like. An alkenyl group may include one, two, three, four, or more carbon-carbon double bonds. For example, C₁₀ alkenyl may include one or more double bonds.

Unless otherwise specified, an alkenyl group described herein refers to both unsubstituted and substituted alkenyl groups.

As used herein, the term “alkynyl” or “alkynyl group” means a linear or branched hydrocarbon including two or more carbon atoms (e.g., two, three, four, five, six, seven, eight, nine, ten, or more carbon atoms) and at least one carbon-carbon triple bond, which is optionally substituted. The notation “C₂-C₁₀ alkynyl” means an optionally substituted linear or branched hydrocarbon including 2-10 carbon atoms and at least one carbon-carbon triple bond, “C₂-C₆alkynyl” means an optionally substituted linear or branched hydrocarbon including 2-6 carbon atoms and at least one carbon-carbon triple bond. An alkynyl group may include one, two, three, four, or more carbon-carbon triple bonds. For example, C₁₀ alkynyl may include one or more carbon-carbon triple bonds. Unless otherwise specified, an alkynyl group described herein refers to both unsubstituted and substituted alkynyl groups.

As used herein, the term “alkoxy” or “alkoxyl group” means a chemical substituent of formula —OR, where R is an alkyl group as defined herein (e.g., C₁-C₁₀ alkyl, C₁₋₆ alkyl or C₁-C₃ alkyl), unless otherwise specified. Exemplary alkoxy groups include methoxy, ethoxy, propoxy (e.g., n-propoxy and isopropoxy), t-butoxy, and the like.

As used herein, the term “aryl” refers to an optionally substituted monocyclic, or fused polycyclic, aromatic carbocycle (i.e., a ring structure having ring atoms that are all carbon). The aryl group may have from 6-10 atoms per ring, denoted C₆₋₁₀ aryl. Examples of suitable aryl groups may include, but are not limited to, phenyl, naphthyl, phenanthryl. As used herein, the term “aryl” is also intended to encompass optionally substituted partially saturated bicyclic aromatic carbocyclic moiety in which a phenyl and a cycloalkyl or cycloalkenyl group are fused together to form a cyclic structure, such as tetrahydronaphthyl, indenyl or indanyl. The aryl group may be a terminal group or a bridging group.

A reference to a percentage (%) content throughout this specification is to be taken to mean a percentage by weight, denoted “% w/w” or “wt. %”. Unless otherwise specified, any reference to a percentage by weight of a component of a composition or formulation described herein refers to the percentage by weight of the specified component with respect to the total components of the composition or formulation. Certain components of the topical compositions described herein may be provided as a liquid. In such cases, it may be more convenient to measure the amount of a liquid component as a volume (e.g., in mL). A skilled person will be able to readily determine the weight of a liquid component given its volume by multiplying the volume (in mL) by the density of the liquid (in g/mL), for example, as measured at 25° C.

The terms “composition” and “formulation” are used interchangeably throughout this specification and have the same meaning.

By hereby reserving the right to proviso out or exclude any individual members of any such group, including any sub-ranges or combinations of sub-ranges within the group, which can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

The reference to any prior art in this specification is not, and should not be taken as, an acknowledgment or any form of suggestion that that prior art forms part of the common general knowledge.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The present invention relates to compositions, particularly topical compositions, comprising a cannabinoid, extracellular matrix component or extracellular matrix fragment, and a pharmaceutically acceptable carrier, excipient, diluent, reagent, or combinations thereof. Such topical compositions may be useful, for example, in the treatment of skin disorders, including dermatological diseases. The present inventors have previously found that certain compositions comprising cannabinoids, such as cannabidiol and isomers or analogs thereof are suitable for anti-aging and skin tone lightening (WO2019/140357) and treatment of dermatological diseases (WO 2021/003488). Advantageously, the topical compositions of the present invention comprising an extracellular matrix component or an extracellular matrix fragment may aid in the dermal and/or transdermal delivery of cannabinoids, e.g., by enhancing the absorption of the cannabinoids into the skin (as measured, for example, by Franz cell diffusion). The topical compositions described herein may also be suitable for improving (e.g., whitening or lightening) skin tone, appearance, texture and/or structure, reducing fine lines and/or wrinkles, firming and/or lifting of the skin, promoting collagen production, reducing signs of aging, moisturizing skin, reducing skin roughness, and the like. The use of topical compositions comprising naturally-derived ingredients, such as the topical compositions described herein, may also have a reduced incidence of side effects compared to existing front-line treatments for skin disorders.

Cannabinoids for use in the topical compositions of the present invention may include any of a broad class of compounds that interact with cannabinoid receptors (including CB1 and CB2 receptors, or any endocannabinoid receptor, including endocannabinoid receptor(s) yet to be discovered) and encompass endocannabinoids (produced naturally in the body by animals), including cannabinoids selected from: the phytocannabinoids (found in cannabis and some other plants), and synthetic cannabinoids (manufactured artificially). A skilled person can readily determine whether a compound interacts with cannabinoid receptor using routine screening assays, such as the ligand binding assays described by Devane et al. (1988) Mol Pharmacol 34:605-613 or by in silico prediction of CB1 affinity (see, e.g., Paulke et al. (2016) Toxicoly Letters 245:1-6).

Exemplary cannabinoids include, but are not limited to, cannabinoids selected from: tetrahydropyran analogs, such as, Δ⁹-tetrahydrocannabinol, Δ⁸-tetrahydrocannabinol, 6,6,9-trimethyl-3-pentyl-6H-dibenzo[b,d]pyran-1-ol, 3-(1,1-dimethylheptyl)-6,6a7,8,10,10a-hexahydro-1-1hydroxy-6,6-dimethyl-9H-dibenzo[b,d]pyran-9-ol, (−)-(3S,4S)-7-hydroxy-delta-6-tetrahydrocannabinol-1,1-dimethylheptyl, (+)-(3S,4S)-7-hydroxy-Δ-6-tetrahydrocannabinol, and Δ⁸-tetrahydrocannabinol-11-oic acid, piperidine analogs, such as, (−)-(6S,6aR,9R,10aR)-5,6,6a,7,8,9,10,10a-octahydro-6-methyl-1-3-[(R)-1-methyl-4-phenylbutoxy]-1,9 phenanthridinediol 1-acetate), aminoalkylindole analogs, such as, (R)-(+)-[2,3-dihydro-5-methyl-3-(4-morpholinylm-ethyl)-pyrrolo[1,2,3,-de]-1,4-benzoxazin-6-yl]-1-naphthalenyl-methanone, open pyran-ring analogs, such as, 2-[3-methyl-6-(1-methylethenyl-2-cyclohexen-1-yl]-5-pentyl-1,3-benzendi-ol, and 4-(1,1-dimethylheptyl)-2,3′-dihydroxy-6′-α-(3-hydroxypropyl)-1′,-2′,3′,4′,5′,6′-hexahydrobiphenyl, lipophilic alkylamides, such as, dodeca-2E,4E,8Z,10E/Z-tetraenoic-acid-isobutylamide, cannabinoid mimetics, salts, solvates, metabolites, metabolic precursors of these compounds and combinations thereof. In some exemplary topical compositions, the cannabinoids may be derived (isolated) from plants including hemp, Echinacea purpurea, Echinacea angustifolia, Acmella oleracea, Helichrysum umbraculigerum, Radula marginata, and combinations thereof and/or oils made from these plants. In other examples, the cannabinoids may be selected from cannabinoids that are manufactured, chemically synthesized, or combinations thereof.

The exemplary cannabinoids that are identified above may be present in a topical composition as described herein in any suitable form. For example, some exemplary topical compositions may contain an individual cannabinoid stereoisomer, whereas some exemplary topical compositions may contain a combination of cannabinoid stereoisomers. For example, in some exemplary topical compositions, the cannabinoid may be cannabidiol (2-(6-isopropenyl-3-methyl-5-cyclohexen-1-yl)-5-pentyl-1,3-benzenediol) or any isomer or stereoisomer of cannabidiol, which has 7 double bonds and 30 stereoisomers; all of the foregoing may be suitable for use in the topical compositions of the present invention. Similarly, some exemplary topical compositions may comprise the various isomers and stereoisomers of cannabidiol (CBD), cannabigerol (CBG), cannabinol (CBN), cannabichromene (CBC), Δ9-tetrahydrocannabivarin (THCV), and cannabivarin (CBV), cannabidivarin (CBDV), cannabinodiol (CBND), cannabielsion (CBE), cannabicyclol (CBL), cannabitriol (CBT), cannabidolic acid (CBDA) and/or other cannabinoids not explicitly identified.

The topical compositions of the present invention may comprise a single (individual), target cannabinoid, e.g., a target cannabinoid that is isolated or substantially free of other (non-target) cannabinoids and/or active agents, including plant-isolated cannabinoids. A single, target cannabinoid may be obtained, for example, from cold-pressed industrial hemp. Cannabinoids are a natural constituent of hemp oil, and different varieties of hemp contain various cannabinoids in different concentrations. A single, target cannabinoid can, for example, be extracted from the hemp oil. The extraction process may separate individual cannabinoids from other cannabinoids to produce a highly purified or isolated single, target cannabinoid for use in topical compositions of the present invention. The extraction process may separate individual cannabinoids from other active agents present in the hemp oil, such as omega-3 and omega-6 fatty acids, antioxidants, and the like. The highly purified or isolated single, target cannabinoid may be the major constituent of the total cannabinoids present in the compositions described herein. In some exemplary topical compositions, the single, target cannabinoid may be isolated or substantially isolated, meaning that it comprises about 85 w/v % to about 100 w/v %, or about 90 w/v % to about 100 w/v %, or about 95 w/v % to about 100 w/v %, or about 98 w/v % to about 100 w/v %, or about 100 w/v % of the total cannabinoids present in the topical compositions. In other exemplary topical compositions, other non-target cannabinoids may make up minor fractions of the cannabinoids present in the topical compositions. For example, the single, target cannabinoid may comprise at least about 85 w/v %, or at least about 90 w/v %, or at least about 95 w/v %, or at least about 98 w/v % of the total cannabinoids present in the topical compositions.

In an embodiment, the single, target cannabinoid comprises about 90 w/v % and one or more other non-target cannabinoids may make up about 10 w/v % of the total cannabinoids present in a topical composition of the present invention.

In some exemplary topical compositions, cannabidiol (CBD) may be the single, target cannabinoid and may be present at from about 30 w/v % to about 100 w/v % of the total cannabinoids present in the topical compositions. In some embodiments, cannabidiol (CBD) may be present at from about 50 w/v % to about 100 w/v %, at from about 75 w/v % to about 100 w/v %, at from about 80 w/v % to about 100 w/v %, or at from about 90 w/v % to about 100 w/v % of the total cannabinoids present in the topical compositions. In some exemplary topical compositions, cannabigerol (CBG) may be the single, target cannabinoid and may be present at from about 30 w/v % to about 100 w/v % of the total cannabinoids present in the topical compositions. In some exemplary topical compositions, cannabigerol (CBG) may be present at from about 50 w/v % to about 100 w/v %, from about 75 w/v % to about 100 w/v %, from about 80 w/v % to about 100 w/v %, or from about 90 w/v % to about 100 w/v % of the total cannabinoids present in the topical compositions. In still other exemplary topical compositions, another single, target cannabinoid, such as cannabinol (CBN), cannabichromene (CBC), Δ9-tetrahydrocannabivarin (THCV), and cannabivarin (CBV), cannabidivarin (CBDV), cannabinodiol (CBND), cannabielsion (CBE), cannabicyclol (CBL), cannabitriol (CBT), cannabidolic acid (CBDA) or other cannabinoids not explicitly identified individually, but not in combination, may be present at from about 30 w/v % to about 100 w/v % of the total cannabinoids present in the topical compositions, and in some exemplary topical compositions, the single cannabinoid may be present at from about 50 w/v % to about 100 w/v %, from about 75 w/v % to about 100 w/v %, from about 80 w/v % to about 100 w/v %, or from about 90 w/v % to about 100 w/v % of the total cannabinoids present in the topical compositions.

In some exemplary topical compositions, two or more single (individual), target cannabinoids may be combined to form a mixture of the single, target cannabinoids prior to addition to the topical compositions, or they may individually be added to the topical compositions. Using single, target cannabinoids, the person of ordinary skill in the art may be able to create compositions or mixtures in which a combination of cannabinoids are present in precise amounts. Hemp oil and other extracts containing cannabinoids may contain widely varying concentrations of individual cannabinoids. Consequently, the effects of the target cannabinoid may be reduced or inhibited by other non-target cannabinoids, making compositions containing hemp oil or hemp extracts less suitable or unsuitable for their intended purpose. Compositions or mixtures containing two or more single, target cannabinoids allow the person of ordinary skill in the art to tailor the composition for specific purposes taking into consideration the effect of each cannabinoid in the mixture on the patient and the impact of each single, target cannabinoid on the other single, target cannabinoids in the composition or mixture.

In some exemplary topical compositions, the cannabinoids present in the compositions may be cannabinoid analogs. The term “cannabinoid analogs” refers to synthetically produced compounds that are structurally similar, but not structurally identical, to a target cannabinoid. A skilled person can readily determine whether a compound that is structurally similar, but not structurally identical, to a target cannabinoid interacts with cannabinoid receptor using routine screening assays, such as the ligand binding assays described by Devane et al. (1988) Mol Pharmacol 34:605-613. Various cannabinoid analogs are known in the art and some exemplary topical compositions of the present invention may comprise such cannabinoid analogs. For example, PCT Publication WO2017/132526 and U.S. Pat. No. 6,630,507, which are each hereby incorporated by reference in their entireties, describe various analogs of cannabidiol. Some exemplary analogs of cannabidiol that may be of use in the present invention may be of general Formula I:

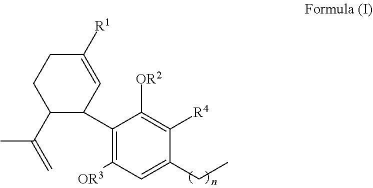

where R¹ is selected from hydrogen, methyl, linear or branched C₂-C₁₀ alkyl, linear or branched C₂-C₁₀ alkenyl, linear or branched C₂-C₁₀ substituted alkyl, and linear or branched C₂-C₁₀ substituted alkenyl, R² and R³ are each independently selected from hydrogen, methyl, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ acyl, linear or branched C₂-C₁₀ substituted acyl, an amine (e.g., NR⁵R⁶, wherein R⁵ and R⁶ are each independently selected from C₁-C₁₀ alkyl) or amino acid (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, homoserine, and norleucine), an amino acid ester, R⁴ is hydrogen, substituted or unsubstituted alkyl (e.g., C₁-C₁₀ alkyl), carboxyl, alkoxy (e.g., —O—C₁-C₁₀ alkyl), aryl (e.g., C₆-C₁₀ aryl), aryloxy (e.g., —O—C₁-C₁₀ aryloxy), arylalkyl (e.g., —C₁-C₁₀ alkyl-C₁-C₁₀ aryl), halo (e.g., F, Cl, Br, I) or amino (e.g., NR⁵R⁶, wherein R⁵ and R⁶ are each independently selected from C₁-C₁₀ alkyl), and n may be an integer from 2 to 10 and the like, and salts and solvates thereof. In some exemplary analogs of cannabidiol, R² and R³ may, independently, be a linear or branched, substituted or unsubstituted C₂-C₁₀ acyl having a carboxylic acid terminus thereby producing a dicarboxylic acid, and salts thereof. Like cannabidiol, cannabidiol analogs can have various isomers. Exemplary isomers for use in the topical compositions of the present invention include all isomers and stereoisomers of such cannabidiol analogs.

In some exemplary topical compositions, cannabinoid analogs such as those described above, may be combined with one or more naturally occurring cannabinoids selected from cannabidiol (CBD), cannabigerol (CBG), cannabinol (CBN), cannabichromene (CBC), Δ9-tetrahydrocannabivarin (THCV), and cannabivarin (CBV), cannabidivarin (CBDV), cannabinodiol (CBND), cannabielsion (CBE), cannabicyclol (CBL), cannabitriol (CBT), cannabidolic acid (CBDA), or other cannabinoids not explicitly identified, to produce a mixture of cannabidiol and cannabidiol analogs. As discussed above, using single, target cannabinoids or cannabinoid analogs, the person of ordinary skill in the art can create compositions or mixtures in which a combination of cannabinoids are present in precise amounts, allowing the person of ordinary skill in the art to tailor the composition for specific purposes taking into consideration the effect of each cannabinoid or cannabinoid analog in the mixture on the patient and the impact of each single, target cannabinoid or cannabinoid analog on the other single, target cannabinoids or cannabinoid analogs in the composition or mixture.

In certain exemplary topical compositions, the single, target cannabinoid or one of the two or more single target cannabinoids may be cannabidolic acid (CBDA). Without wishing to be bound by theory, CBDA may exhibit improved hydrophilicity over other isomers of cannabidiol, which may allow for improved solubility and delivery of CBDA to the skin. Suitable CBDA may be modified, partially digested, or otherwise acted upon by enzymes in the skin to produce for example cannabidiol (CBD), which may be the active form cannabidiol in exemplary topical compositions of the present invention. Thus, CBDA may act as a prodrug in some exemplary topical compositions. Other cannabidiol analogs or isomers may produce a similar prodrug effect and are also contemplated by the present invention. The term “prodrug” is used in its broadest sense and encompasses those derivatives that are converted in vivo to the active agent (i.e., a target cannabinoid). Such derivatives would readily occur to those skilled in the art.

Exemplary topical compositions of the present invention may comprise up to about 50 w/w % cannabinoid (including cannabidiol, cannabinoid analogs, isomers of cannabinoids, stereoisomers of cannabinoids, and combinations thereof), relative to the total weight of the composition. Some exemplary topical compositions may comprise from about 50 w/w % to about 0.5 w/w % cannabinoid, relative to the total weight of the compositions, from about 30 w/w % to about 1 w/w % cannabinoid, relative to the total weight of the compositions, from about 20 w/w % to about 1 w/w % cannabinoid, relative to the total weight of the composition, from about 20 w/w % to about 5 w/w % cannabinoid, relative to the total weight of the composition, or any range or individual concentrations encompassed by these example ranges (e.g., about 1 w/w %, 5 w/w %, 10 w/w %, 15 w/w %, 20 w/w %, 30 w/w %, 40 w/w %, or 50 w/w % cannabinoid). In particular exemplary topical compositions, the cannabinoid component may be present at from about 15 w/w % to about 10 w/w %, relative to the total weight of the composition, or any range or individual concentrations encompassed by this example range (e.g., about 10 w/w %, 11 w/w %, 12 w/w %, 13 w/w %, 14 w/w %, 15 w/w % cannabinoid). In some exemplary topical compositions, the cannabinoid component may be one single (individual), target cannabinoid that is isolated or highly purified, and in other exemplary topical compositions, the cannabinoid component may be two or more single, target cannabinoids that are individually isolated or highly purified. In still other exemplary topical compositions, the cannabinoid component may contain a de minimis amount of one or more non-target cannabinoids, for example, less than about 10 w/w %, less than about 5 w/w %, less than about 2 w/w %, less than about 1 w/w %, or less than about 0.5 w/w % non-target cannabinoids.

The cannabidiol in some exemplary topical compositions may be isolated cannabidiol, or oils, solvents, and emulsions containing cannabidiol. For example, some exemplary topical compositions, may include cannabidiol derived from hempseed oil. Hempseed oil is generally manufactured from varieties of Cannabis sativa that do not contain significant amounts of tetrahydrocannabinol (THC), the psychoactive element of the cannabis plant. This manufacturing process typically includes cleaning the seed to 99.99% before pressing the oil. Hempseed oil generally also contains omega-6 and omega-3 fatty acids. For example, about 30-35% of the weight of hempseed oil are essential fatty acids (EFAs), i.e., linoleic acid, omega-6 (LA, 55%), α-linolenic acid, omega-3 (ALA, 22%), γ-linolenic acid, omega-6 (GLA, 1-4%) and stearidonic acid, omega-3 (SDA, 0-2%). Thus, some exemplary topical compositions may contain fatty acids such as omega-6 and omega-3 fatty acids.

Exemplary oils that may be suitable for use in the topical compositions of the present invention may include cannabidiol oil and various plant derived oils containing cannabidiol, such as, hempseed oil, Echinacea purpurea, Echinacea angustifolia, Acmella oleracea, Helichrysum umbraculigerum, Radula marginata, and the like. In some exemplary topical compositions, cannabidiol isolated from such plants or made synthetically may be formulated with an oil selected from olive oil, grapeseed oil, tea tree oil, almond oil, avocado oil, sesame oil, evening primrose oil, sunflower oil, kukui nut oil, jojoba oil, walnut oil, peanut oil, pecan oil, macadamia nut oil, coconut oil, and the like, and combinations thereof.

Topical compositions in accordance with the present invention comprise an extracellular matrix component, an extracellular matrix fragment, or a combination thereof. The use of an extracellular matrix component or an extracellular matrix fragment in combination with a cannabinoid in topical compositions as described herein may advantageously increase the absorption of the cannabinoid into (or across) the skin relative to compositions without an extracellular matrix component or an extracellular matrix fragment. Increased absorption of the cannabinoid may result in a greater proportion of the cannabinoid applied to the skin being delivered to the site of action, the cannabinoid being delivered to the site of action faster, or both. Increased absorption may be measured, for example, using Franz cell diffusion, infrared spectroscopy, mass spectrometry and fluorescein labeling, or the like.

The extracellular matrix is a network of proteins and other molecules, such as polysaccharides and proteoglycans, that surround and support cells and tissues in the body. Thus, exemplary extracellular matrix proteins suitable for use in the compositions of the present invention may comprise an extracellular matrix protein, an extracellular matrix polysaccharide (e.g., hyaluronic acid), an extracellular matrix proteoglycan, a fragment thereof, or combinations thereof. Suitable extracellular matrix proteins may include, but are not limited to, elastin, collagen, fibronectin, lectin, laminin, merosin, tenascin, vitronectin and fibrillin. Suitable extracellular matrix polysaccharides may include, but are not limited to, hyaluronic acid, heparin sulfate, chondroitin sulfate and keratan sulfate. In some embodiments, the extracellular matrix may comprise an extracellular matrix polysaccharide covalently bonded to an extracellular matrix protein (e.g., a proteoglycan, such as syndecan). Other suitable extracellular matrix components and extracellular matrix fragments will be apparent to those skilled in the art and may include, for example, integrins and discoidin domain receptors.

The extracellular matrix components or fragments contained in exemplary topical compositions may vary from composition to composition and may include components selected from hyaluronic acid, chondroitin sulfate, keratan sulfate, elastin, collagen, fibronectin, lectin, laminin, merosin, tenascin, vitronectin, fibrillin, syndecan and fragments thereof, and combinations thereof. In some exemplary topical compositions, the extracellular matrix components may be extracellular matrix fragments including, without limitation, those selected from hyaluronic acid fragments, collagen fragments, fibronectin fragments, elastin fragments, lectin fragments, and combinations thereof. It is to be understood that such extracellular matrix fragments are to be selected so as to retain at least a portion of the function of the extracellular matrix component from which it is derived. In certain exemplary topical compositions, the extracellular matrix fragments may have a particular size, e.g., molecular weight or distribution that, without wishing to be bound by theory, may impact the delivery of components of the composition into and through the various layers of the epidermis. Extracellular matrix components and extracellular matrix fragments suitable for use in the present invention may include natural, synthetic and semi-synthetic extracellular matrix components and fragments, and may include one or more chemical modifications to alter one or more physical properties of the component or fragment (e.g., solubility in aqueous or non-aqueous media).

Suitable extracellular matrix components, extracellular matrix fragments, and combinations thereof in exemplary topical compositions may have specific average molecular weights. For example, suitable extracellular matrix components, extracellular matrix fragments, and combinations thereof may have an average molecular weight of from about 2,000 Da to about 100,000 Da, from about 2,000 Da to about 60,000 Da, from about 2,000 Da to about 50,000 Da, from about 2,000 Da to about 40,000 Da, from about 2,000 Da to about 30,000 Da, from about 2,000 to about 20,000 Da, from about 2,000 to about 15,000 Da, from about 2,000 Da to about 10,000 Da, from about 5,000 Da to about 40,000 Da, less than about 60,000 Da, less than about 50,000 Da, less than about 40,000 Da, less than about 30,000 Da, less than about 20,000 Da, less than about 15,000 Da, less than about 10,000 Da, less than about 5,000 Da, about 60,000 Da, about 50,000 Da, about 40,000 Da, about 30,000 Da, about 20,000 Da, about 15,000 Da, about 12,500 Da, about 10,000 Da, about 8,500 Da, about 7,500 Da, about 5,000 Da, about 2,000 Da to about 5,000 Da, about 5,000 Da to about 10,000 Da, about 10,000 Da to about 20,000 Da, about 20,000 Da to about 30,000 Da, about 30,000 Da to about 40,000 Da, about 20,000 Da to about 40,000 Da, about 40,000 Da to about 60,000 Da, or about 60,000 Da to about 100,000 Da or any range or individual number falling within these aforementioned exemplary ranges and molecular weights.

In some exemplary topical compositions, the extracellular matrix component, extracellular matrix fragment, and combination thereof may comprise hyaluronic acid or a fragment thereof. Hyaluronic acid interacts with, for example, CD44, receptor for hyaluronic acid-mediated motility (RHAMM), and intercellular adhesion molecule-1 (ICAM-1). Hyaluronic acid is a polymer of disaccharides that disrupts intercellular interactions between cells allowing passage of components of the exemplary topical compositions through the epidermis. In some exemplary topical compositions, the hyaluronic acid or fragments thereof may be cross-linked, and in other exemplary topical compositions, hyaluronic acid or fragments thereof may not be cross-linked. In still other exemplary topical compositions, a portion of the hyaluronic acid or fragments thereof may be cross-linked, and another portion of the hyaluronic acid or fragments thereof may not be cross-linked. The hyaluronic acid and fragments thereof of various exemplary topical compositions, whether individual or cross-linked, may have an average molecular weight of less than about 60,000 Da, from about 2,000 Da to about 60,000 Da, from about 2,000 Da to about 40,000 Da, from about 5,000 Da to about 40,000 Da, from about 2,000 Da to about 30,000 Da, from about 2,000 Da to about 20,000 Da, or from about 2,000 Da to about 10,000 Da.

In some exemplary topical compositions, the extracellular matrix component, extracellular matrix fragment, or combination thereof may comprise collagen or a fragment thereof. Collagen can be isolated in various forms and from several sources. Exemplary collagens that may be suitable for use in the present invention include those selected from collagen type I, collagen type II, collagen type III, collagen type IV, or collagen type V. The collagen may be fibrillary collagen or non-fibrillar collagen. Low molecular weight collagens can be made, for example, by hydrolysis and, like hyaluronic acid, low molecular weight collagen may disrupt cell-cell and cell-scaffold attachments by interrupting intercellular interactions. Suitable collagen and fragments thereof may have an average molecular weight of less than about 60,000 Da, from about 2,000 Da to about 60,000 Da, from about 2,000 Da to about 40,000 Da, from about 5,000 Da to about 40,000 Da, from about 2,000 Da to about 30,000 Da, from about 2,000 Da to about 20,000 Da, or from about 2,000 Da to about 10,000 Da.

In certain exemplary topical compositions, the extracellular matrix component, extracellular matrix fragment, or combination thereof may comprise fibronectin or a fragment thereof. Fibronectin is a protein dimer, consisting of two nearly identical monomers linked by a pair of disulfide bonds. Fibronectin binds to membrane-spanning receptor proteins called integrins and extracellular matrix components such as collagen, fibrin, and heparin sulfate proteoglycans. Like hyaluronic acid and collagen, fibronectin or fragments thereof may disrupt cell-cell and cell-scaffold attachments by interrupting intercellular interactions. Suitable fibronectin and fragments thereof may have an average molecular weight of less than about 60,000 Da, from about 2,000 Da to about 60,000 Da, from about 2,000 Da to about 40,000 Da, from about 5,000 Da to about 40,000 Da, from about 2,000 Da to about 30,000 Da, from about 2,000 Da to about 20,000 Da, or from about 2,000 Da to about 10,000 Da.

In some exemplary topical compositions, the extracellular matrix component, extracellular matrix fragment, or combination thereof may comprise elastin or a fragment thereof. Elastin is a protein found in connective tissue and allows many tissues in the body to resume their shape after stretching or contracting. Like hyaluronic acid, collagen, and fibronectin, elastin fragments may disrupt cell-cell and cell-scaffold attachments by interrupting intercellular interactions. Elastin fragments can be obtained commercially or may be generated by protease digestion, such as using proteinase K or thermolysin. For example, commercially available Elastin E91 preparation from Protein Preparations, Inc., St. Louis, Mo., is a suitable elastin product to subject to digestion, having a molecular weight of from about 1,000 to about 60,000 Dalton. Additionally, a series of digests available under the trade name ProK, and specifically ProK-60 and ProK-60P, which are elastin peptide mixtures derived from the proteolytic digestion of insoluble elastin derived from bovine neck ligaments, may also be used. Suitable elastin and fragments thereof may have an average molecular weight of less than about 60,000 Da, from about 2,000 Da to about 60,000 Da, from about 2,000 Da to about 40,000 Da, from about 5,000 Da to about 40,000 Da, from about 2,000 Da to about 30,000 Da, from about 2,000 Da to about 20,000 Da, or from about 2,000 Da to about 10,000 Da.

In some exemplary topical compositions, the elastin fragments may include amino acid sequences selected from GAAPG, GVVPG, GGGPG, GLLPG, GIIPG, GSSPG, GTTPG, GCCPG, GMMPG, GFFPG, GYYPG, GWWPG, GDDPG, GNNPG, GEEPG, GQQPG, GRRPG, GHHPG, GKKPG, GPPPG, G3Hyp3HypPG (Glycine-3-hydroxyproline-3-hydroxyproline-Proline-Glycine), G4Hyp4HypPG (Glycine-4-hydroxyproline-4-hydroxyproline-Proline-Glycine), RRPEV, QPSQPGGV, PGGV, GPGV, KPGV, GPGL, EGSA, PGGF, GGGA, KPGKV, PGGV, KPKA, GPGGV, GPQA, GGPGI, PGPGA, GPGGV, GQPF, GGKPPKPF, GGQQPGL, GGPGI, VGVAPG, IGVAPG, PGGVLPG, VGVVPG, IGLGPGGV, VGAMPG, VGLSPG, IGAMPG, IGLSPG, GVAPGV, VAPGVG, APGVGV, PGVGVA, GVGVAP, and combinations thereof.

In some exemplary topical compositions, the extracellular matrix component, extracellular matrix fragment, or combination thereof may comprise laminin or a fragment thereof. The protein laminin is a complex, consisting of three different polypeptide chains (α, β, γ) that are bound to each other by disulfide bonds into a cross-shaped molecule comprising one long and three short arms with globules at each end. The α-2 chain is a subunit of laminin-2 (merosin) and laminin-4 (S-merosin). Its cell binding ability (via membrane bound integrin receptors) makes laminin an effective substrate coating for stimulating and enhancing cell migration and neurite outgrowth. Suitable laminin and fragments thereof may have an average molecular weight of less than about 60,000 Da, from about 2,000 Da to about 60,000 Da, from about 2,000 Da to about 40,000 Da, from about 5,000 Da to about 40,000 Da, from about 2,000 Da to about 30,000 Da, from about 2,000 Da to about 20,000 Da, or from about 2,000 Da to about 10,000 Da.

In some exemplary topical compositions, the extracellular matrix component, extracellular matrix fragment, or combination thereof may comprise lectin or a fragment thereof. Lectins are often complex, multi-domain, multimeric proteins. However, the carbohydrate-binding activity of mammalian lectins is normally the property of a carbohydrate recognition domain or CRD. The CRDs of mammalian lectins fall into three phylogenetically conserved classes: C-type, S-type and P-type. C-type lectins require Ca++ for ligand binding, are extracellular membrane and soluble proteins and, as a class, bind a variety of carbohydrates. S-type lectins are most active under reducing conditions, occur both intra- and extracellularly, bind β-galactosides and do not require Ca++. P-type lectins bind mannose 6-phosphate as their primary ligand. Suitable lectin and fragments thereof may have an average molecular weight of less than about 60,000 Da, from about 2,000 Da to about 60,000 Da, from about 2,000 Da to about 40,000 Da, from about 5,000 Da to about 40,000 Da, from about 2,000 Da to about 30,000 Da, from about 2,000 Da to about 20,000 Da, or from about 2,000 Da to about 10,000 Da.

In some exemplary topical compositions, the extracellular matrix component, extracellular matrix fragment, or combination thereof may comprise at least one decoy molecule selected from heparin sulfate, chondroitin sulfate, keratan sulfate, laminin, merosin, tenascin, vitronectin, and fibrillin, and fragments thereof and combinations thereof. These decoy molecules may have an average molecular weight of less than about 60,000 Da, about 2,000 Da to about 60,000 Da, from about 2,000 Da to about 40,000 Da, from about 5,000 Da to about 40,000 Da, from about 2,000 Da to about 30,000 Da, from about 2,000 Da to about 20,000 Da, or from about 2,000 Da to about 10,000 Da.

In some exemplary topical compositions, the extracellular matrix component, extracellular matrix fragment, or combination thereof may comprise an extracellular matrix receptor, such as those selected from integrins, discoidin domain receptors and syndecans, and fragments thereof and combinations thereof. These decoy molecules may have an average molecular weight of less than about 60,000 Da, from about 2,000 Da to about 60,000 Da, from about 2,000 Da to about 40,000 Da, from about 5,000 Da to about 40,000 Da, from about 2,000 Da to about 30,000 Da, from about 2,000 Da to about 20,000 Da, or from about 2,000 Da to about 10,000 Da.

The size of the extracellular matrix component, extracellular matrix fragment, or combination thereof may impact delivery of various components of exemplary topical compositions to the skin. Therefore, in some exemplary topical compositions, the extracellular matrix component or extracellular matrix fragment may have a size, e.g., molecular weight, falling within specific ranges as follows:
Very small: extracellular matrix component or extracellular matrix fragment having an average molecular weight of from about 2,000 Da to about 5,000 Da;
Small: extracellular matrix component or extracellular matrix fragment having an average molecular weight of from about 5,000 Da to about 20,000 Da;
Mid-sized: extracellular matrix component or extracellular matrix fragment having an average molecular weight of from about 20,000 Da to about 40,000 Da;
Large: extracellular matrix component or extracellular matrix fragment having an average molecular weight of from about 40,000 Da to about 60,000 Da; and
Very large: extracellular matrix component or extracellular matrix fragment having an average molecular weight of from about 60,000 Da to about 100,000 Da.
   Because extracellular matrix components or extracellular matrix fragments do not generally have a strictly uniform size, individual exemplary topical compositions may include extracellular matrix component or extracellular matrix fragment having an average molecular weight falling outside the “average molecular weight.” For example, the decoy molecule may include individual molecules that are large and extra-large or very small and small when the average molecular weight is mid-sized. In some embodiments, the topical compositions of the present invention comprise small (e.g., about 5,000 Da to about 20,000 Da) and/or very small (e.g., about 2,000 Da to about 5,000 Da) extracellular matrix components or extracellular matrix fragments.

The amount of extracellular matrix components, extracellular matrix fragments, or combinations thereof in topical compositions of the present invention may modulate the depth of penetration of the components of the compositions. In general, the amount of extracellular matrix components, extracellular matrix fragments, or combinations thereof present in exemplary topical compositions may be from about 0.001 wt. % to about 10 wt. %, and in particular exemplary topical compositions, the amount of decoy in such compositions may be from about 0.1 wt. % to about 2.0 wt. %, from about 0.25 wt. % to about 3.0 wt. %, from about 0.5 wt. % to about 5.0 wt. %, from about 0.75 wt. % to about 7.5 wt. %, or any range or individual concentrations encompassing these example ranges. In some exemplary topical compositions, extracellular matrix components, extracellular matrix fragments, or combinations thereof may be present at from about 0.001 wt. % to about 10 wt. %, from about 0.001 wt. % to about 9 wt. %, from about 0.001 wt. % to about 8 wt. %, from about 0.001 wt. % to about 7 wt. %, from about 0.001 wt. % to about 6 wt. %, from about 0.001 wt. % to about 5 wt. %, from about 0.001 wt. % to about 4 wt. %, from about 0.001 wt. % to about 3 wt. %, from about 0.001 wt. % to about 3 wt. %, or from about 0.001 wt. % to about 1 wt. % of the total composition. Specific examples include about 0.001 wt. %, about 0.01 wt. %, about 0.1 wt. %, about 0.5 wt. %, about 1 wt. %, about 2 wt. %, about 5 wt. %, about 10 wt. %, and ranges between any two of these values.

In some exemplary topical compositions, extracellular matrix components, extracellular matrix fragments, or combinations thereof may be present at from about 1 microgram to about 100 μmilligrams per mL of the total compositions, at from about 1 microgram to about 10 milligrams per mL of the total compositions, from about 1 microgram to about 5 μmilligrams per mL of the total compositions, from about 1 microgram to about 1 μmilligram per mL of the total compositions, or from about 1 microgram to about 100 micrograms per mL the total compositions.

Some exemplary topical compositions may include more than one size class of extracellular matrix components or extracellular matrix fragments. For example, a first portion of the extracellular matrix component or extracellular matrix fragment of some exemplary topical compositions may comprise small or very small extracellular matrix components or extracellular matrix fragments, and a second portion of the extracellular matrix component of the compositions may comprise large or very large extracellular matrix components or extracellular matrix fragments. The presence of a first portion and a second portion of extracellular matrix components or extracellular matrix fragments having different sizes may further improve delivery of the components of the skin. Without wishing to be bound by theory, the larger extracellular matrix component or extracellular matrix fragment may aid in dispersion of the smaller extracellular matrix component or extracellular matrix fragment and reduce the likelihood that the smaller extracellular matrix component or extracellular matrix fragment aggregate at pores in the skin blocking channels to deeper layers of the epidermis. The concentration of the first portion of extracellular matrix component or extracellular matrix fragment and the second portion of extracellular matrix component or extracellular matrix fragment may be the same or different. For example, the concentration of the first portion of extracellular matrix component or extracellular matrix fragment may be from about 0.001 wt. % to about 10 wt. %, from about 0.1 wt. % to about 2.0 wt. %, from about 0.25 wt. % to about 3.0 wt. %, from about 0.5 wt. % to about 5.0 wt. %, from about 0.75 wt. % to about 7.5 wt. %, or any range or individual concentrations encompassing these example ranges, and the concentration of the second portion of extracellular matrix component or extracellular matrix fragment may be equal to the concentration of the first portion of extracellular matrix or extracellular matrix fragment components in the range of from about 0.001 wt. % to about 10 wt. %, from about 0.1 wt. % to about 2.0 wt. %, from about 0.25 wt. % to about 3.0 wt. %, from about 0.5 wt. % to about 5.0 wt. %, from about 0.75 wt. % to about 7.5 wt. %, or any range or individual concentrations encompassing these example ranges. In other exemplary topical compositions, the concentration of the first portion of extracellular matrix component or extracellular matrix fragment may be from about 0.001 wt. % to about 10 wt. %, from about 0.1 wt. % to about 2.0 wt. %, from about 0.25 wt. % to about 3.0 wt. %, from about 0.5 wt. % to about 5.0 wt. %, from about 0.75 wt. % to about 7.5 wt. %, or any range or individual concentrations encompassing these example ranges, and the concentration of the second portion of extracellular matrix component or extracellular matrix fragment may be less than the first extracellular matrix component or extracellular matrix fragment and within the range of from about 0.001 wt. % to about 10 wt. %, from about 0.1 wt. % to about 2.0 wt. %, from about 0.25 wt. % to about 3.0 wt. %, from about 0.5 wt. % to about 5.0 wt. %, from about 0.75 wt. % to about 7.5 wt. %, or any range or individual concentrations encompassing these example ranges. In still other exemplary topical compositions, the concentration of the first portion of extracellular matrix component or extracellular matrix fragment may be from about 0.001 wt. % to about 10 wt. %, from about 0.1 wt. % to about 2.0 wt. %, from about 0.25 wt. % to about 3.0 wt. %, from about 0.5 wt. % to about 5.0 wt. %, from about 0.75 wt. % to about 7.5 wt. %, or any range or individual concentrations encompassing these example ranges, and the concentration of the second portion of extracellular matrix component or extracellular matrix fragment may be greater than the first extracellular matrix component or extracellular matrix fragment and within the range of from about 0.001 wt. % to about 10 wt. %, from about 0.1 wt. % to about 2.0 wt. %, from about 0.25 wt. % to about 3.0 wt. %, from about 0.5 wt. % to about 5.0 wt. %, from about 0.75 wt. % to about 7.5 wt. %, or any range or individual concentrations encompassing these example ranges. In such exemplary topical compositions, the first portion of extracellular matrix component or extracellular matrix fragment may have a smaller average molecular weight than the average molecular weight of the second portion of the extracellular matrix component or extracellular matrix fragment.

Unless indicated otherwise, the term “therapeutically effective amount” is not particularly limited, so long as the cannabinoid component and the extracellular matrix component or extracellular matrix fragment are present in an amount effective for treating a skin disorder, such as a dermatological disease, or for improving (e.g., whitening or lightening) skin tone, appearance, texture and/or structure, reducing fine lines and/or wrinkles, firming and/or lifting of the skin, promoting collagen production, reducing signs of aging, moisturizing skin, reducing skin roughness, and the like. The therapeutically effective amount of the cannabinoid component and the extracellular matrix components, extracellular matrix fragments, or combinations thereof, combined or individually, may be from about 2 milligrams per kilogram (mg/kg) to about 100 mg/kg, from about 2 mg/kg to about 50 mg/kg, from about 2 mg/kg to about 25 mg/kg, or any range or individual concentrations encompassed by these example ranges, wherein mg refers to the mass or weight of the cannabinoid component and the extracellular matrix component or extracellular matrix fragment, combined or individually, and kg refers to the mass or weight of the patient in need of treatment. The therapeutically effective amount of exemplary topical compositions that are administered topically may vary by the area of the skin to which the compositions are administered, i.e., the “administration area,” and by the amount of the compositions that are applied to the administration area. In general, a therapeutically effective amount of a topical administration of exemplary topical compositions may be from about 0.1 μmilligram per square centimeter (mg/cm²) to about 50 mg/cm², from about 0.5 mg/cm² to about 25 mg/cm², from about 0.5 mg/cm² to about 10 mg/cm², from about 0.5 mg/cm² to about 5 mg/cm², from about 0.75 mg/cm² to about 2 mg/cm², or any range or individual concentrations encompassed by these example ranges, wherein mg refers to the mass or weight of the composition and cm² refers to the surface area of the skin of the patient in need of treatment to which an exemplary composition is applied.

Topical compositions in accordance with the present invention further comprise a pharmaceutically acceptable carrier, excipient, diluent, reagent, or combinations thereof. Where a carrier, excipient, diluent and/or reagent is used, they must be “pharmaceutically acceptable” in the sense of being compatible with the other ingredients of the composition and not injurious to the subject. Such pharmaceutically acceptable carriers, excipient, diluents and/or reagents will be apparent to those skilled in the art and may depend on the intended mode of administration. For example, the carriers, excipients, diluents and/or reagents may vary depending on the formulation and/or mode of administration. In some embodiments, the compositions may be provided in sustained-release formulations.

Exemplary topical compositions may be combined with various additional agents to improve efficacy of treatment, aid in delivery of the cannabinoid component and the extracellular matrix component or extracellular matrix fragment contained therein, treat additional symptoms associated with a skin disorder, such as dermatological disease, or for improving (e.g., whitening or lightening) skin tone, appearance, texture and/or structure, reducing fine lines and/or wrinkles, firming and/or lifting of the skin, promoting collagen production, reducing signs of aging, moisturizing skin, reducing skin roughness, and the like.

For example, some exemplary topical compositions may include one or more bioenhancers. Bioenhancers include any compound or composition that aids in the transport of another compound across epithelial membranes. Non-limiting examples of suitable bioenhancers may include P-glycoprotein inhibitors, compounds that reverse P-glycoprotein-mediated efflux, limit metabolism of active agents, increase gastric emptying time and intestinal motility, reduce degradation of the active agent by hydrochloric acid, modify cell membrane permeability, produce a cholagogue effect, modify the bioenergetics and thermogenic properties of the active agent, suppress first pass metabolism, and inhibit metabolizing enzymes, stimulate gamma glutamyl transpeptidase, enhance the uptake of amino acids, and the like, and combinations thereof. In some exemplary topical compositions, the bioenhancers may be herbal and/or nutraceutical bioenhancers. Further examples of suitable bioenhancers may include piperidine, quercetin, genistein, naringin, sinomenine, glycyrrhizin, nitrile glycosides cuminum cyminum, zingiver officinale, lysergol, allium sativum, aloe vera, and the like and combinations thereof. In some exemplary topical compositions, the bioenhancers may be selected from liposomes, microspheres, nanoparticles, transferosomes, ethosomes, nanoemulsions, microemulsions, lipid-based systems, polymeric micelle formulations, ketoprofen-loaded solid lipid nanoparticles, and the like, which may be made from beeswax, carnauba wax, or other natural waxes and solid lipids, and combinations thereof. In some exemplary topical compositions, the bioenhancers may be liposomal enhancers including, for example, those selected from Ginkgo biloba lipid-based systems, silybin lipid-based systems, ginseng lipid-based systems, hawthorn lipid-based systems, quercetin lipid-based systems, curcumin lipid-based systems, and the like and combinations thereof. In further exemplary topical compositions, the bioenhancers may be selected from capsaicin transferosomes, colchicine tranferosomes, vincristine tranferosomes, and the like and combinations thereof, which may find particular use as natural skin penetration agents.

The amount of bioenhancer in exemplary topical compositions of the present invention may be from about 0.05% to about 20% (w/w), relative to the total amount of the compositions, from about 0.1% to about 10% (w/w), relative to the total weight of the compositions, from about 0.1% to about 5% (w/w), relative to the total weight of the compositions, from about 0.1% to about 2% (w/w), relative to the total weight of the compositions, or any range or individual concentrations encompassed by these example ranges.

By way of example, piperidine may enhance bioavailability by modulating DNA receptor binding and cell signal transduction, while inhibiting efflux pumps that remove the active agent from cells; this may inhibit drug metabolizing enzymes and stimulate absorption by stimulating gut amino acid transporters and inhibiting cellular pumps responsible for drug elimination from cells and intestinal production of glucuronic acid. Piperidine may also increase the absorption of the active agent in the gastrointestinal tract and may inhibit enzymes responsible for drug metabolism especially in the liver during first pass metabolism such as hepatic arylhydrocarbon hydrolase and UDP-glucuronyltransferase activities. Piperidine may modify the rate of glucuronidation by lowering the endogenous UDP-glucuronic acid content and also by inhibiting transferase activity. Piperidine may inhibit P-glycoprotein and cytochrome P450 3A4, also CYP1A1, CYP1B1, CYP1B2, CYP2E1, CYP3A4, among others and make target receptors more responsive to drugs, acting as receptors for drug molecules, increasing GIT vasculature by vasodilation to increase the absorption of drugs, modulation of cell membrane dynamics which increases transport of drugs across the cell membranes.

Some exemplary topical compositions may further include a vitamin. Suitable vitamins may include, but are not limited to, vitamin B1, vitamin B2, vitamin B3, vitamin B5, vitamin B6, vitamin B9, vitamin B12, lipoic acid, vitamin A, biotin, vitamin K, vitamin C, vitamin D, vitamin E, and combinations thereof.

Some exemplary topical compositions may further include hydrocortisone or any steroid within Groups I to VII in the US classification system. Group I steroids that may be suitable for use in the topical composition of the present invention include, but are not limited to, those selected from clobetasol propionate, betamethasone dipropionate, halobetasol, and diflorasone diacetate. Suitable Group II steroids may include, but are not limited to, those selected from fluocinonide, halcinonide, amcinonide, and desoximetasone. Suitable Group III steroids may include, but are not limited to, those selected from triamcinolone acetonide, mometasone furoate, fluticasone propionate, betamethasone dipropionate, and halometasone. Suitable Group IV steroids may include, but are not limited to, those selected from fluocinolone acetonide, hydrocortisone valerate, hydrocortisone butyrate, flurandrenolide, triamcinolone acetonide, and mometasone furoate. Suitable Group V steroids may include, but are not limited to, those selected from fluticasone propionate, desonide, fluocinolone acetonide, and hydrocortisone valerate. Suitable Group VI steroids may include, but are not limited to, those selected from alclometasone dipropionate, triamcinolone acetonide, fluocinolone acetonide, and desonide. Suitable Group VII steroids may include, but are not limited to, those selected from hydrocortisone (2.5%) and hydrocortisone (1%). The amount of hydrocortisone or steroid in the exemplary topical compositions may be from about 0.01% to about 5% (w/w), relative to the total weight of the compositions, or in some exemplary topical compositions, from about 0.1% to about 1% (w/w), relative to the total weight of the compositions, or any range or individual concentrations encompassed in these example ranges.

Some exemplary topical compositions may further include an anti-inflammatory compound selected from methotrexate, tofacitinib, 6-mercaptopurine, azathioprine sulphasalazine, mesalazine, olsalazine chloroquinine/hydroxychloroquinine, penicillamine, aurothiomalate (intramuscular and oral), azathioprine, colchicine, corticosteroids (oral, inhaled, and local injection), a beta-2 adrenoreceptor agonist (salbutamol, terbutaline, salmeteral), xanthine (theophylline, aminophylline), cromoglycate, nedocromil, ketotifen, ipratropium and oxitropium, cyclosporin, FK506, rapamycin, mycophenolate mofetil, leflunomide, an NSAID (e.g. ibuprofen), a corticosteroid (e. g. prednisolone), a phosphodiesterase inhibitor, an adenosine agonist, an antithrombotic agent, a complement inhibitor, an adrenergic agent, an agent that interferes with signaling by proinflammatory cytokines such as TNF or IL-1 (e.g., a NIK, IKK, p38 or MAP kinase inhibitor), an IL-1 converting enzyme inhibitor, a T-cell signaling inhibitor (e.g. a kinase inhibitor), a metalloproteinase inhibitor, sulfasalazine, a 6-mercaptopurine, an angiotensin converting enzyme inhibitor, a soluble cytokine receptor (e.g. soluble p55 or p75 TNF receptors and the derivatives p75TNFRigG (etanercept) and p55TNFRigG (Lenercept), siL-1RI, siL-1RII, siL-6R), an anti-inflammatory cytokine (e.g. IL-4, IL-1 0, IL-11, IL-13 and TGF), celecoxib, folic acid, hydroxychloroquine sulfate, rofecoxib, etanercept, infliximab, adalimumab, certolizumab, tocilizumab, abatacept, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, cortisone, betamethasone, morphine sulfate, lidocaine hydrochloride, indomethacin, glucosamine sulf/chondroitin, amitriptyline HCl, sulfadiazine, oxycodone HCV acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-ILlS, BIRB-796, SCIO-469, VX-702, AMG-548, VX-740, Roflumilast, IC-485, CDC-801, SlPI agonists (such as FTY720), a PKC family inhibitor (e.g. Ruboxistaurin or AEB-071) or Mesopram, budenoside; epidermal growth factor; a corticosteroid; cyclosporin, sulfasalazine; an aminosalicylate; 6-mercaptopurine; azathioprine; metronidazole; a lipoxygenase inhibitor; mesalamine; olsalazine; balsalazide; an antioxidant; a thromboxane inhibitor; an IL-1 receptor antagonist; an anti-IL-1 monoclonal antibody; an anti-IL-6 monoclonal antibody; a growth factor; an elastase inhibitor; a pyridinyl-imidazole compound; an antibody to or antagonist of other human cytokines or growth factors (e.g. TNF, LT, IL-1, IL-2, IL-6, IL-7, IL-8, IL-12, IL-15, IL-16, IL-23, EMAP-II, GM-CSF, FGF, and PDGF); a cell surface molecule (e.g. CD2, CD3, CD4, CD8, CD25, CD28, CD30, CD40, CD45, CD69, or CD90 or their ligands); methotrexate; cyclosporine; FK506; rapamycin; mycophenolate mofetil; leflunomide; an NSAID (e.g. ibuprofen); a TNF converting enzyme inhibitor; therapeutic agents that target an intrinsic checkpoint blockade, such as, for example, the gene encoding Cytokine-inducible SH2-containing protein (CISH), antibody BGB-A317, Nivolumab, or Pembrolizumab, atezolizumab, avelumab, durvalumab, ipilimumab, and the like and combinations thereof. The amount of anti-inflammatory in exemplary topical compositions may be from about 0.01% to about 5% (w/w), relative to the total weight of the compositions, or from about 0.1% to about 1% (w/w), relative to the total weight of the compositions, or any range or individual concentrations encompassed by these example ranges.

Some exemplary topical compositions may further include an antibiotic or antifungal agent. The antibiotic compound is not particularly limited, and may be selected from antibacterial, antifungal, antiprotozoal, and other antimicrobial agents. In certain exemplary topical compositions, the antibiotic may be selected from ampicillin, bacampicillin, carbenicillin indanyl, mezlocillin, piperacillin, ticarcillin, amoxicillin-clavulanic acid, ampicillin-sulbactam, benzylpenicillin, cloxacillin, dicloxacillin, methicillin, oxacillin, penicillin G, penicillin V, piperacillin tazobactam, ticarcillin clavulanic acid, nafcillin, procaine penicillin, cefadroxil, cefazolin, cephalexin, cephalothin, cephapirin, cephradine, cefaclor, cefamandol, cefonicid, cefotetan, cefoxitin, cefprozil, ceftmetazole, cefuroxime, loracarbef cefdinir, ceftibuten, cefoperazone, cefixime, cefotaxime, cefpodoxime proxetil, ceftazidime, ceftizoxime, ceftriaxone, cefepime, azithromycin, clarithromycin, clindamycin, dirithromycin, erythromycin, lincomycin, troleandomycin, cinoxacin, ciprofloxacin, enoxacin, gatifloxacin, grepafloxacin, levofloxacin, lomefloxacin, moxifloxacin, nalidixic acid, norfloxacin, ofloxacin, sparfloxacin, trovafloxacin, oxolinic acid, gemifloxacin, perfloxacin, imipenem-cilastatin, meropenem, aztreonam, and the like, and combinations thereof. Suitable antifungal agent antibiotics may include amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox, flucytosine, griseofulvin, haloprogin, tolnaftate, undecylenic acid, crystal violet, balsam of Peru, and the like and combinations thereof. The amount of the antibiotic or antifungal agent in exemplary topical compositions may be from about 0.01% to about 5% (w/w), relative to the total weight the compositions, or from about 0.1% to about 1% (w/w), relative to the total weight of the compositions, or any range or individual composition encompassed by these example ranges.

Some exemplary topical compositions may further include an analgesic agent. The analgesic agent is not particularly limited and may include, for example, methyl salicylate, codeine, morphine, methadone, pethidine, buprenorphine, hydromorphine, levorphanol, oxycodone, fentanyl, a non-steroidal anti-inflammatory drug, and the like and combinations thereof. The amount of the analgesic agent in exemplary topical compositions may be from about 0.01% to about 5% (w/w), relative to the total weight of the compositions, or from about 0.1% to about 1% (w/w), relative to the total weight of the compositions, or any range or individual concentrations encompassed by these example ranges.

Some exemplary topical compositions may further include an antiviral compound.

The anti-viral compound is not particularly limited and may include, for example, anti-viral compounds selected from acyclovir, famciclovir, penciclovir, valacyclovir, trifluridine, docosanol, amantadine, rimantadine, oseltamivir, and zanamivir. The amount of the anti-viral compound in exemplary topical compositions may be from about 0.01% to about 5% (w/w), relative to the total weight of the compositions or from about 0.1% to about 1% (w/w), relative to the total weight of the compositions, or any range or individual concentrations encompassed by these example ranges.

Cannabinoids are generally insoluble in water. Exemplary topical compositions may therefore include one or more pharmaceutically acceptable solvents or co-solvents for at least partially solubilizing the cannabinoid. The solvent or co-solvent may be present in an amount effective to have the cannabinoids substantially solubilized in the exemplary topical compositions. Therefore, the amount of a particular solvent in exemplary topical compositions may vary based on the type, amount, and/or concentration of the cannabinoid contained therein. The amount of a particular solvent may also vary based on the partition coefficient of the particular cannabinoid molecule.

In certain exemplary topical compositions, the solvents may be pharmaceutically acceptable organic solvents selected from dehydrated alcohol, ethanol, propanol, isopropanol, propylene glycol, polyethylene glycol, and the like, and combinations thereof. The amount of organic solvent in an exemplary topical composition may vary based on the intended formulation and the solubility of the cannabinoid. The amount of dehydrated alcohol in the exemplary topical compositions may be in a range of from about 10% to about 90% (w/w), from about 15% to about 65% (w/w), from about 15% to about 50% (w/w) relative to the total weight of the compositions, or any range or independent concentration encompassed by these example ranges.

Some exemplary topical compositions may include a co-solvent such as, for example, polyethylene glycol. In certain exemplary topical compositions, the co-solvent may be a low molecular weight polyethylene glycol such as polyethylene glycol 400 (PEG 400). The co-solvent may be present in exemplary topical compositions at a concentration from about 1% to about 50% (w/w), from about 1% to about 15% (w/w), or from about 1% to about 10% (w/w), relative to the total weight of the compositions, or any range or independent concentration encompassed by these example ranges. In various exemplary topical compositions, the total solvent and co-solvents may be present at a concentration from about 65% (w/w) to about 75% (w/w) relative to the total weight of the compositions.

Some exemplary topical compositions may further include a solubilizing agent. Solubilizing agents suitable for use in the topical compositions of the present invention may include, for example, those selected from Capryol 90, Cremophor RH40, Labrafil M 1944 CS, Labrafil M 2125 CS, Lauroglycol 90, PEG MW>4000, Plurol Oleique CC 497, poloxamer 124, poloxamer 188, Softigen 701, Softigen 767, Tagat TO, Tween 80, triacetin, triethylcitrate, tributylcitrate, acetyl triethylcitrate, acetyl tributyl citrate, ethyl oleate, ethyl caprylate, ethyl butyrate, triacetin, 2-pyrrolidone, 2-piperidone, N-methylpyrrolidone, N-ethylpyrrolidone, N-hydroxyethyl pyrrolidone, N-octylpyrrolidone, N-laurylpyrrolidone, dimethylacetamide, Miglyol, lanolin, petrolatum, mineral oil, and the like, and combinations thereof. The solubilizing agent may be present at a concentration from about 5% to about 85% (w/w) of the total inactive ingredients present in the topical compositions disclosed herein.

Other components such as preservatives, antioxidants, surfactants, absorption enhancers, viscosity modifiers, bulking agents, diluents, coloring agents, and/or pH modifiers may also be incorporated into exemplary topical compositions of the present invention. The amount of each of these components may be optimized for each formulation, to obtain a stable product (dosage form) having the desired shelf-life. Generally, suitable formulations may include from about 0.001% to about 20% (w/w) of a pharmaceutically acceptable preservative, antioxidant, surfactant, absorption enhancer, viscosity modifier, bulking agent, diluent, coloring agent, or pH modifier, relative to the total weight of the formulation.

Some exemplary topical compositions may include an effective (stabilizing) amount of one or more pharmaceutically acceptable antioxidants. Any known antioxidants may be used to stabilize exemplary topical compositions, including but not limited to antioxidants selected from butylated hydroxyanisole (BHA), butylated hydroxytoluene (BHT), propyl gallate, lecithin, Vitamin E tocopherol, sesamin, sesamol, sesamolin, alpha tocopherol, ascorbic acid, ascorbyl palmitate, fumaric acid, malic acid, sodium ascorbate and sodium metabisulphite, as well as chelating agents such as disodium EDTA. Exemplary topical compositions may also include antioxidant synergists to prevent oxidative degradation. Such antioxidant synergists may include, for example, disodium edetate or the like. Such exemplary topical compositions may include from about 0.001% to about 20% (w/w) antioxidant, from about 0.001 to about 1% (w/w), from about 0.01% to about 0.1% (w/w) relative to the total weight of the compositions, or any range or individual concentrations encompassed by these example ranges.

Some exemplary topical compositions may further include one or more organic or inorganic bases. Examples of suitable organic bases may include, but are not limited to, those selected from any pharmaceutically acceptable primary, secondary, and tertiary organic amines, such as methanolamine, ethanolamine, meglumine, other alkylamines (e.g. di-alkyl amines and tri-alkyl amines), and the like, and combinations thereof. Suitable inorganic bases may include, for example, those selected from sodium hydroxide (NaOH), magnesium hydroxide (MgOH) and the like, and combinations thereof. In such exemplary topical compositions, the organic or inorganic bases may be present at a concentration of from about 0.001% to about 20% (w/w), from about 0.01% to about 15% (w/w), from about 0.01% to about 5% (w/w), from about 0.05% to about 2% (w/w) of the total weight of the compositions, or any range or individual value encompassed by these example ranges. Some exemplary topical compositions may include both an organic base and an inorganic base.

Some exemplary topical compositions may include a preservative. Preservatives typically have bactericidal and fungicidal properties. For example, parabens are widely used preservatives in the pharmaceutical industry. Some exemplary topical compositions may include a combination of parabens which may allow for the use of lower concentrations of preservative with increasing preservative activity. Examples of suitable parabens may include, for example, those selected from methyl paraben, propyl paraben, and the like, and combinations thereof. Various exemplary topical compositions may include from about 0.001% to about 5.0% (w/w), from about 0.001% to about 1.0% (w/w), from about 0.01% to about 0.05% (w/w) paraben, relative to the total weight of the compositions, or any range or individual concentrations encompassed by these example ranges. Some exemplary topical compositions may include a combination of parabens, such as from about 0.001% to about 0.1% (w/w) methyl paraben and from about 0.001% to about 0.1% (w/w) propyl paraben, relative to the total weight of the compositions, or any range or individual concentrations of methyl or propyl paraben encompassed by these ranges.

Some exemplary topical compositions may contain chemical preservatives such as, for example, those selected from cetylpyridinium chloride, K-Sorbate, Na-Benzoate, various parabens, other chemical preservatives, and combinations thereof. Such chemical preservatives may be present at a concentration of from about 0.001 wt. % to about 5.0 wt. %, from about 0.001 wt. % to about 1.0 wt. %, from about 0.01 wt. % to about 0.05 wt. %, relative to the total weight of the compositions, or any range or individual concentrations encompassed by these example ranges.

Other suitable additives for use in exemplary topical compositions of the present invention may include, for example, those selected from sodium compounds and copper-based compounds. In particular, sodium containing compounds have been linked to elastogenesis and may enhance the anti-aging effects of compositions containing cannabinoids and extracellular matrix components, extracellular matrix fragments, or combinations thereof.

Some exemplary topical compositions may further include a physiologically acceptable buffer, for example, to maintain the pH of the compositions at or within a physiologically compatible pH range, enhance the solubility and stability of the cannabinoid, and the like. Suitable buffers may include, but are not limited to those selected from acetate, bicarbonate, citrate, phosphate, pharmaceutically acceptable salts thereof and combinations or mixtures thereof. Exemplary topical compositions may include from about 0.1% to about 20% (w/w), from about 0.5% to about 10% (w/w), relative to the total weight of the compositions, or any range or individual concentrations encompassed by these example ranges. The concentration of buffer may be such that the compositions have a pH of from about 5 to about 10, from about 6 to about 8, or about 7.

Certain exemplary topical compositions may be isotonic. Isotonic formulations may be provided by the addition of a tonicity agent. Suitable tonicity agents include, but are not limited to, those selected from any pharmaceutically acceptable sugar such as dextrose, salt, i.e., sodium chloride, and the like, and combinations or mixtures thereof. Exemplary topical compositions may have a concentration of tonicity agent of from about 100 mOsm/kg to about 500 mOsm/kg, from about 200 mOsm/kg to about 400 mOsm/kg, from about 250 mOsm/kg to about 300 mOsm/kg, or any range or individual concentrations encompassed by these example ranges.

Some exemplary topical compositions may further include a viscosity modifier such as, for example, a viscosity modifier selected from cellulose or cellulose derivatives such as ethylcellulose, hydroxyethylcellulose, hydroxypropylcellulose, caboxymethylcellulose, sodium hydroxypropylmethylcellulose, methylcellulose, methylethylcellulose, sodium carboxymethylcellulose, Aerosil (silicon dioxide), cetostearyl alcohol, cetyl alcohol, stearyl alcohol, Gelucires 33/01, 39/01 and 43/01, glyceryl behenate (Compritol 888 A TO), glyceryl palmitostearate (Precirol AT05), Softisans 100, 142, 378 and 649, stearyl alcohol carbomer, xanthan gum, maltodextrin, acacia, tragacanth, povidone and polyvinyl alcohol. The concentration of viscosity modifier may be from about 0.01% to about 25% (w/w), from about 0.01% to about 10% (w/w), from about 0.01% to about 5% (w/w), relative to the total weight of the compositions, or any range or individual concentrations encompassed by these example ranges.

Some exemplary topical compositions may further include an absorption enhancer such as, for example, those selected from Gelucire 44/14, Gelucire 50/13, Tagat TO, Tween 80, isopropyl myristate, polysorbates, sorbitan esters, poloxamer block copolymers, PEG-35 castor oil, PEG-40 hydrogenated castor oil, caprylocaproyl macrogol-8 glycerides, PEG-8 caprylic/capric glycerides, sodium lauryl sulfate, dioctyl sulfosuccinate, polyethylene lauryl ether, ethoxydiglycol, propylene glycol mono-di-caprylate, glycerol monocaprylate, glyceryl fatty acids (C8-C18) ethoxylated, oleic acid, linoleic acid, glyceryl caprylate/caprate, glyceryl monooleate, glyceryl monolaurate, caprylic/capric triglycerides, ethoxylated nonylphenols, PEG-(8-50) stearates, olive oil PEG-6 esters, triolein PEG-6 esters, lecithin, d-alpha tocopheryl polyethylene glycol 1000 succinate, polycarbonate, sodium glycocholate, sodium taurocholate, cyclodextrins, citric acid, sodium citrate, triacetin, and the like and combinations thereof. In some exemplary topical compositions, the absorption enhancer may be triacetin. The absorption enhancer in the exemplary topical compositions may be present at a concentration of from about 0.01% to about 25% (w/w), from about 0.01% to about 10% (w/w), from about 0.01% to about 5% (w/w) relative to the total weight of the compositions, or any range or individual concentrations encompassed by these example ranges.

Some exemplary topical compositions may include a bulking agent such as, for example, a bulking agent selected from microcrystalline cellulose, mannitol, xylitol, starches, and the like and combinations thereof. In such exemplary topical compositions, the bulking agent may be present at a concentration of from about 0.01% to about 25% (w/w), from about 0.01% to about 10% (w/w), from about 0.01% to about 5% (w/w), relative to the total weight of the compositions, or any range or individual concentrations encompassed by these example ranges.

Some exemplary topical compositions may contain a gelling or suspension agent such as, for example, a gelling or suspension agent selected from carbomers such as Carbopol, modified cellulose derivatives, naturally-occurring, synthetic or semi-synthetic gums such as xanthan gum, acacia and tragacanth, modified starches, co-polymers such as those formed between maleic anhydride and methyl vinyl ether, colloidal silica and methacrylate derivatives sold under the trade name Eudragit™, and the like and combinations thereof. In some exemplary topical compositions, the gelling or suspension agent may be present at a concentration of from about 0.01% to about 25% (w/w), from about 0.01% to about 10% (w/w), from about 0.01% to about 5% (w/w), relative to the total weight of the compositions, or any range or individual concentrations encompassed by these example ranges.

In some exemplary topical compositions, additional excipients may be incorporated into the compositions of the invention as needed, including, for example, excipients selected from surfactants (e.g. Capryol 90; Cremophor RH40; Gelucire 44/14; Gelucire 50/13; Imwitor 91; Imwitor 308; Imwitor 380; Imwitor 742; Imwitor 780K; Imwitor 928; Imwitor 988; Labrafil M 1944 CS; Labrafil M 2125 CS; Lauroglycol 90; Tagat TO; Tween 80; and mixtures thereof); and emulsifiers (e.g., Gelucire 44/14; Gelucire 50/13; Imwitor 91; Imwitor 308; Imwitor 380; Imwitor 742; Imwitor 780K; Imwitor 928; Imwitor 988; poloxamer 124; poloxamer 188; Tagat TO; Tween 80; lecithin; lysolecithin; phosphatidylcholine; phosphatidylethanolamine; phosphatidylglycerol; phosphatidic acid; phosphatidylserine; lysophosphatidylcholine; lysophosphatidylethanolamine; lysophosphatidylglycerol; lysophosphatidic acid; lysophosphatidylserine; PEG-phosphatidylethanolamine; PVP-phosphatidylethanolamine; sodium lauryl sulfate, and mixtures thereof).

Some exemplary topical compositions may further include an anti-acne agent. The anti-acne agent is not particularly limited and may, for example, be at least one anti-acne agent selected from the group consisting of salicylic acid and benzoyl peroxide. The total amount of the anti-acne compound(s) in exemplary topical compositions is not particularly limited, so long as it is a therapeutically effective amount. For example, the total amount of acne agent(s) may be from about 0.01% (w/w) to about 5% (w/w), relative to the total weight of the exemplary topical compositions, or from about 0.1% (w/w) to about 1% (w/w), relative to the total weight of the exemplary topical compositions.

Some exemplary topical compositions may include a humectant, which can be referred to as a soothing, smoothing, moisturizing or protective agent. The humectant is not particularly limited and may, for example, be at least one humectant selected from calamine, dodecylsulphate, sodium lauryl sulphate (SLS), a polyoxyethylene ester of polysorbitan, such as monooleate, monolaurate, monopalmitate, monostearate esters, esters of sorbitan, the polyoxyethylenes ethers, the sodium dioctylsulphosuccinate (DOSS), lecithin, and sodium docusate. Some exemplary topical compositions may comprise sodium lauryl sulphate, calamine, or combinations thereof. The amount of the humectant in exemplary topical compositions is not particularly limited, so long as it is a therapeutically effective amount. Exemplary amounts present in topical compositions of the present invention may be from about 0.01% (w/w) to about 5% (w/w), relative to the total weight of exemplary topical compositions, or from about 0.1% (w/w) to about 1% (w/w), relative to the total weight of the exemplary topical compositions.

Some exemplary topical compositions may contain a UV-absorbing compound, which can be referred to as a sunscreen agent. The UV-absorbing compound is not particularly limited and may, for example, be at least one UV-absorbing compound selected from glyceryl PABA, padimate 0, roxadimate, dioxybenzone, oxybenzone, sulisonbenzone, octocrylene, octyl methoxycinnamate, ethoxyethyl p-methoxycinnamate, homomenthyl salicylate, ethylhexyl salicylate, trolamine salicylate, avobenzone, ecamsule, ensulizole, bemotrizinol, and bisoctrizole. The amount of the UV-absorbing compound in exemplary topical compositions is not particularly limited, so long as it is a therapeutically effective amount. Exemplary amounts present in topical compositions of the present invention may be from about 0.01% (w/w) to about 5% (w/w), relative to the total weight of the exemplary topical compositions, or from about 0.1% (w/w) to about 1% (w/w), relative to the total weight of the exemplary topical compositions.

Other additives conventionally used in pharmaceutical compositions may be included in exemplary topical compositions, and these additives are well known in the art. Such additives may include, for example, those additives selected from pharmaceutically acceptable detackifiers, anti-foaming agents, chelating agents, viscomodulators, tonicifiers, colorants, odorants, opacifiers, suspending agents, binders, fillers, plasticizers, lubricants, and mixtures thereof. The amounts of such additives suitable for use in the topical compositions of the present invention can be readily determined by one skilled in the art, according to the particular properties desired, keeping in mind the possibility that ideally, the presence of any such additives will have no negative impact on the stability of the final formulations.

Suitable coloring agents for use in exemplary topical compositions may be selected, for example, from red, black, and yellow iron oxides and FD&C dyes such as FD&C Blue No. 2, FD&C Red No. 40, and the like.

It is recognized that pharmaceutical excipients may perform more than one function and are therefore characterized as having different uses depending on the particular application. While the use of an excipient in the context of an exemplary topical composition may determine the function of the excipient, the inclusion of any particular excipient into any one or more categories as set forth above is not meant to limit the function of that excipient.

Although the ingredients of the exemplary topical compositions are characterized herein as percentage based on weight, one skilled in the art will appreciate that scaled-up versions of the exemplary topical compositions specifically described herein may be characterized instead on a volume percentage basis. Where the density of a particular component is 1 g/ml the amount of the component based on volume and weight will be the same. Where the density deviates from 1 g/ml, the amounts based on weight or volume will differ accordingly.

The exemplary topical compositions discussed above may be formulated as, for example, lotions, creams, soaps, shampoos, balms and the like and may be designed, for example, for treating a dermatological disease and/or for moisturizing, anti-aging, or anti-wrinkle, or treating acne, rough skin, dandruff, eczema, and the like, as well as lightening or whitening skin.

Exemplary topical compositions may be formulated into gels, creams, lotions, or ointments. Gels are semi-solid dispersion of liquid or oil particles in a semi-solid medium and may include, for example, petroleum jelly and coco butter. In these formulations, the cannabinoids (e.g., cannabidiol or cannabidiol analogs) and extracellular matrix components or extracellular matrix fragments may be in the form of a suspension or the form a gel with the pharmaceutically acceptable carrier, excipient, diluent, reagent, or combinations thereof, and may be mixed with solids such as starches and methyl cellulose. Creams refer to semi-solid emulsions of oil and water in approximately equal proportions. They are divided into two types: oil-in-water (O/W) creams, composed of small droplets of oil dispersed in a continuous phase; and water-in-oil (W/O) creams, composed of small droplets of water dispersed in a continuous oily phase. Creams can provide a barrier to protect the skin. Lotions are low- to medium-viscosity topical preparation. Most lotions are oil-in-water emulsions containing an emulsifier such as cetyl alcohol to prevent separation of these two phases, and include, for example, fragrances, glycerol, petroleum jelly, dyes, preservatives, proteins, and stabilizing agents. Ointments are compositions in which oil and water are provided in a ratio of from 7:1 to 2:1, from 5:1 to 3:1, or 4:1. Ointments are generally formulated using oils, waxes, water, alcohols, petroleum products, water, and other agents to prepare formulations with various viscosities and solvent properties. Commonly used ointment formulations include those ointment formulations selected form oleaginous base (White Ointment), absorption base, W/O emulsion base (Cold Cream type base), O/W emulsion base (Hydrophilic Ointment), water soluble base, in addition to others. These preparations are used to dissolve or suspend substances or products with medicinal or cosmetic value.

Exemplary topical compositions in liquid form may contain liquid phases in addition to or to the exclusion of water. Exemplary of such additional liquid phases are liquid phases selected from glycerin, vegetable oils such as cottonseed oil, olive oil, grapeseed oil, tea tree oil, almond oil, avocado oil, sesame oil, evening primrose oil, sunflower oil, kukui nut oil, jojoba oil, walnut oil, peanut oil, pecan oil, macadamia nut oil, coconut oil, and the like, and combinations thereof, organic esters such as ethyl oleate, and water-oil emulsions.

Some exemplary topical compositions may be in the form of a tonic. Tonics are extracts that provide a method for oral administration of an herbal component or components to a subject in need of treatment. Tonics may be prepared by mixing an herb, herbs or components or combinations thereof with a suitable solvent wherein a component or components of an herb or herbs or combinations thereof are extracted into a solvent by aid of heating, often heat necessary such that the solvent reaches its boiling temperature, in which the component or components of the herb are reasonably soluble. Suitable tonic solvents for use in the topicals compositions of the present invention may include tonic solvents selected, for example, from pharmacologically acceptable solvents such as organic solvents, water-based solvents, alcohols, and other orally administrable solvents such as, but not limited to, water, purified water, preserved water, vegetable glycerin, propylene carbonate, 3-methoxy-3methyl-1-butanol (MMB), polyethylene glycol, rice bran oil, and combinations thereof.

Some exemplary topical composition may be in the form of a soap, which are formulations that comprise a salt of a fatty acid. Soaps are mainly used as surfactants for washing, bathing, and cleaning, but they are also used in textile spinning and are important components of lubricants. Soaps for cleansing are usually obtained by treating vegetable or animal oils and fats with a strongly alkaline solution. Fats and oils are composed of triglycerides; three molecules of fatty acids are attached to a single molecule of glycerol. The alkaline solution, which is often called lye (although the term “lye soap” refers almost exclusively to soaps made with sodium hydroxide), is believed to promote a chemical reaction known as saponification. In saponification, the fats are first hydrolyzed into free fatty acids, which then combine with the alkali to form crude soap. Glycerol (glycerine) is usually liberated and is either left in or washed out and recovered as a useful byproduct, depending on the process employed.

Some exemplary topical composition may be in the form of a shampoo, which is a hair care product used for the removal of oils, dirt, skin particles, dandruff, environmental pollutants, and other contaminant particles that gradually build up in hair.

Topical compositions of the present invention may be prepared by any method known in the art of pharmacology. In general, such preparatory methods include the steps of bringing the cannabinoid, into association with an extracellular matrix component or extracellular matrix fragment, and one or more pharmaceutically acceptable carriers, excipients, diluents and/or reagents, and any other accessory ingredients and then, if necessary and/or desirable, shaping and/or packaging the product into a desired single- or multi-dose unit. In certain embodiments, unit dosage compositions are those containing a daily dose or unit, daily sub-dose, or an appropriate fraction thereof, of the cannabinoid and extracellular matrix component or extracellular matrix fragment. As used herein, a “unit dose” is a discrete amount of the topical composition comprising a predetermined amount of the active ingredients. The amount of the active ingredients (i.e., the cannabinoid and extracellular matrix component or extracellular matrix fragment) is generally equal to the dosage of the active ingredient that would be administered to a subject and/or a convenient fraction of such a dosage such as, for example, one-half or one-third of such a dosage.

Some exemplary methods may include dispersing the cannabinoids (e.g., cannabidiol or cannabidiol analogs), optionally together with an extracellular matrix component or extracellular matrix fragment and/or one or more pharmaceutically acceptable carriers, excipients, diluents and/or reagents, into mineral oil or silicone oil to obtain an oil phase; dispersing an emulsifier, a thickener; and/or a stabilizer into water in a separate vessel to obtain an aqueous phase; and blending the oil phase and the aqueous phase to form an emulsion. Some exemplary methods may include dispersing the cannabinoids, optionally together with an extracellular matrix component or extracellular matrix fragment, and/or one or more pharmaceutically acceptable carriers, excipients, diluents and/or reagents into the aqueous phase or the emulsion, rather than into the oil phase, of exemplary topical compositions. Some exemplary methods may comprise heating while obtaining an oil phase or dispersing an emulsifier, a thickener and/or a stabilizer into water in a separate vessel to obtain an aqueous phase. Temperatures of this heating are not particularly limited, so long as the oil phase and the aqueous phase result from the dispersing.

Some exemplary methods for making topical compositions of the invention may include mixing an oil phase including the cannabinoids (e.g., cannabidiol or cannabidiol analogs), optionally together with an extracellular matrix component or extracellular matrix fragment and/or one or more pharmaceutically acceptable carriers, excipients, diluents and/or reagents, and an emulsifier with an aqueous phase to form a mixture, and heating said mixture at a temperature of from 45° C. and 85° C. to form an aqueous emulsion. Suitable emulsifiers may include, but are not limited to, emulsifiers selected from cetyl alcohol, stearic acid, and mixtures thereof. The water phase may further include a stabilizing agent such as VEEGUM® and/or CARBOPOL®, and/or may include one or more additives selected form fragrances, glycerol, petroleum jelly, colorants, dyes, preservatives, proteins, and the like, and combinations thereof. Some exemplary methods may produce lotions.

Some exemplary methods for making topical compositions of the invention may include combining a surfactant such as, for example, sodium lauryl sulfate, sodium laureth sulfate, or combinations thereof with a co-surfactant such as cocamidopropyl betaine, in an aqueous phase, optionally comprising an extracellular matrix component or extracellular matrix fragment, and/or one or more pharmaceutically acceptable carriers, excipients, diluents and/or reagents, and mixing the aqueous phase with an oil phase containing the cannabinoids (e.g., cannabidiol or cannabidiol analogs), optionally comprising an extracellular matrix component or extracellular matrix fragment, and/or pharmaceutically acceptable carriers, excipients, diluents and/or reagents, to form a viscous liquid. Some exemplary methods may include adding other additives, such as salt (sodium chloride), a preservative, and/or fragrance, to the aqueous phase.

General considerations in the formulation and/or manufacture of compositions for topical administration, may be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

The topical compositions described herein may be suitable for treating various skin disorders, muscle pain, or arthritic pain. A method of treating various skin disorders, muscle pain, or arthritic pain utilizing exemplary topical compositions as described herein, comprises administering a therapeutically effective amount of a topical composition including a cannabinoid and extracellular matrix components, extracellular matrix fragments, or combinations thereof, to a patient in need of treatment. Topical compositions in accordance with the present invention may be administered in a manner compatible with the dosage formulation in a therapeutically effective amount. Suitable regimes for administration are also variable but are typified by an initial administration followed by repeated doses at one or more time intervals by a subsequent administration. Where a single exemplary topical composition is not available for a treatment, or when application of such a single composition is not desirable, administration of composition may also include the sequential application of several different exemplary topical composition to achieve a desired effect.

“Providing” when used in conjunction with a therapeutic means to administer a therapeutic directly onto a target tissue or to administer a therapeutic to a patient whereby the therapeutic positively impacts the tissue to which it is targeted. Thus, as used herein, the term “providing”, when used in conjunction with a composition as described herein comprising a cannabinoid or, in certain examples, cannabidiol or cannabidiol analogs, may include, but is not limited to, providing a cannabinoid or, in certain examples, cannabidiol or cannabidiol analogs, onto the target tissue. “Providing” a topical composition as described herein may be accomplished by topical administration, optionally in combination with other known techniques. Such combination techniques include heating, radiation and ultrasound.

Heating skin on a patient may, for example, open pores, activate the various mechanisms of a cell, and increase diffusion into said tissue and cells. Thus, exemplary methods and used of the present invention may further comprise heating in connection with providing an exemplary topical composition.

Administration of topical compositions of the present invention includes local administration of the topical compositions to skin of a mammal. Local administration is typically carried out by topical administration of a liquid, gel, cream, ointment, or lotion form of exemplary topical composition(s).

In some embodiments, the present invention provides a method of treating a skin disorder, muscle pain, or arthritic pain, comprising topically administering to a patient in need of treatment a composition comprising a cannabinoid, extracellular matrix component or extracellular matrix fragment, and a pharmaceutically acceptable carrier, excipient, diluent, reagent, or combinations thereof.

In some embodiments, the present invention provides use of a cannabinoid, extracellular matrix component or extracellular matrix fragment, and a pharmaceutically acceptable carrier, excipient, diluent, reagent, or combinations thereof in the manufacture of a medicament for treating a skin disorder, muscle pain, or arthritic pain by topical administration.

In some embodiments, the present invention provides a composition comprising a cannabinoid, extracellular matrix component or extracellular matrix fragment, and a pharmaceutically acceptable carrier, excipient, diluent, reagent, or combinations thereof for treating a skin disorder, muscle pain, or arthritic pain by topical administration.

As described elsewhere herein, skin disorders include dermatological diseases, as well as features of the skin that may be considered undesirable. For example, topical compositions as described herein may improve skin tone, appearance, texture, and/or structure. Such improvements may be obtained by topically administering exemplary topical compositions that contain cannabinoids and extracellular matrix components to skin. Some exemplary topical compositions can be used, for example, for lightening or whitening skin, reducing the signs of aging such as by reducing fine lines and wrinkles, firming and lifting of the skin, and/or promoting generation of collagen. Topical compositions as described herein containing a cannabinoid and an extracellular matrix component, such as hyaluronic acid, may be administered to patients in need of treatment. Such patients may exhibit signs of aging or wish to improve or modify the tone, appearance, texture, and the like of their skin. Topical compositions as described herein containing a cannabinoid and an extracellular matrix component, such as hyaluronic acid, may be administered to a patient in need of treatment, wherein the patient may suffer from disease-causing blemishes on or darkening of the skin, or disorders that affect the tone appearance, texture, and the like of the skin.

Thus, treating a skin disorder as described herein may comprise improving (e.g., whitening or lightening) skin tone, appearance, texture and/or structure, reducing fine lines and/or wrinkles, firming and/or lifting of the skin, promoting collagen production, generally reducing the signs of aging, moisturizing the skin, reducing skin roughness, and combinations thereof. Improving skin tone may include, for example, lightening the color of skin, reducing production of melanin, diminishing the amount of melanin in skin, reducing redness of skin, or combinations thereof. Reducing the signs of aging may include, for example, reducing fine lines, reducing wrinkles, firming skin, lifting skin, and reducing sagging.

Non-limiting examples of targeted dermatological diseases include those selected from eczema, psoriasis, dandruff, sunburn, dermatitis (contact, atopic nummular, and seborrheic), poison ivy and conditions caused by other plant materials containing urushiol or related molecules, type 1 and type 2 herpes, shingles, insect bites, itching, anal itching, vaginal itching, acne, warts, acute and chronic dermatoses, viral skin diseases, bacterial skin diseases, fungal skin diseases, scabies, melanoma, pyoderma, cellulitis, keratinocyte carcinoma, decubitus ulcer, and alopecia areata and veterinary diseases of the skin. Some exemplary methods specifically target psoriasis.

In some embodiments, the topical compositions described herein may be suitable to provide a local analgesic effect, for example, in the treatment of muscle and/or arthritic pain.

The topical compositions of the present invention are to be administered to a subject to deliver an effective amount of the cannabinoid. Suitable effective amounts may depend on the age, gender, weight, and general health of the patient and can be determined by the attending physician. Suitable dosages may lie within the range of about 0.1 ng per kg of body weight to 100 g per kg of body weight per dosage. The cannabinoid dosage may be in the range of 1 μg to 10 g per kg of body weight per dosage, such as is in the range of 1 mg to 1000 mg per kg of body weight per dosage. In one embodiment, the cannabinoid dosage may be in the range of 1 mg to 500 mg per kg of body weight per dosage. In another embodiment, the cannabinoid dosage may be in the range of 1 mg to 250 mg per kg of body weight per dosage. In yet another embodiment, the cannabinoid dosage may be in the range of 1 mg to 200 mg per kg of body weight per dosage, such as up to 50 mg per kg body weight per dosage.

In certain embodiments, an effective amount of the cannabinoid for administration one or more times a day to a 70 kg adult human may comprise about 0.0001 mg to about 4000 mg, about 0.0001 mg to about 3000 mg, about 0.0001 mg to about 200 mg, about 0.001 mg to about 1500 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of the cannabinoid per unit dosage form. In certain embodiments, formulations of the topical composition may be at dosage levels sufficient to deliver the cannabinoid in an amount from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg, of subject body weight per day, one or more times a day, to obtain the desired therapeutic effect. In certain embodiments, an effective amount of a cannabinoid for topical administration to a 70 kg adult human may comprise about 0.0001 mg to about 4000 mg, about 0.0001 mg to about 3000 mg, about 0.0001 mg to about 200 mg, about 0.001 mg to about 1500 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of an extract or compound per unit dosage form. In some embodiments, a single dose may be sufficient to treat a skin disorder, muscle pain or arthritic pain, which may be delivered in one or more aliquots to achieve the desired dose. In other embodiments, multiple doses may be required to treat a skin disorder, muscle pain or arthritic pain. Dosing may occur at intervals of minutes, hours, days, weeks, months, or years or continuously over any one of these periods. The administered amount may be an amount sufficient to treat or alleviate the symptoms associated with a skin disorder, muscle pain or arthritic pain.

The amount of cannabinoid administered per dose or the total volume of composition administered will depend on such factors as the nature and severity of the symptoms, the age, weight, and general health of the patient. It is recognized that relative amounts of extracellular matrix components, extracellular matrix fragments, carriers, excipients, diluents, reagents, and/or any additional ingredients in a pharmaceutical composition as disclosed herein may also depending upon the identity, size, and/or condition of the subject treated, as well as the mode of administration.

Topical compositions as described herein may be administered in a single dose or a series of doses. Suitable dosage amounts and dosing regimens can be determined by the attending physician and may depend on the particular condition being treated, the severity of the condition as well as the general age, health and weight of the subject. It will be appreciated that dose ranges as described herein provide guidance for the administration of provided pharmaceutical compositions to an adult. The amount to be administered can be determined by a medical practitioner or person skilled in the art.

In certain embodiments, it is envisaged that the topical compositions described herein may be administered to a subject in need thereof as a substitute or replacement for other traditional medication for the treatment of a skin disorder, muscle pain or arthritic pain. In other embodiments, it is envisaged that the topical compositions as described herein be administered to a subject in need thereof as a supplement or adjunct to traditional medication. In still other embodiments, it is envisaged that the topical compositions as described herein may be administered to a subject in need thereof in the absence of adjunct therapy. Replacing traditional medication for the treatment of skin disorders, muscle pain, or arthritic pain with topical compositions of the present invention may be advantageous, particularly where the traditional medication is associated with one or more adverse effects.

In other embodiments, topical compositions as described herein may be administered to a subject in need thereof, together with one or more additional therapeutic agents for a discrete period of time, to address specific symptoms of a skin disorder, muscle pain or arthritic pain. In still other embodiments, the subject in need thereof may be treated with topical compositions as described herein and one or more additional therapeutic agents (administered sequentially or in combination) for the duration of the treatment period. Such combination therapy may be particularly useful, for example, where an additive or synergistic therapeutic effect is desired. Where the active agents are provided in separate dosage formulations, the active agents may be administered separately or in conjunction. In addition, the administration of one active agent may be prior to, concurrent with, or subsequent to the administration of the other agent.

The phrase “combination therapy” as used herein, is to be understood to refer to administration of an effective amount, using a first amount of a topical composition as described herein, and a second amount of an additional suitable therapeutic agent. An “effective amount” of the second agent will depend on the type of drug used. Suitable dosages are known for approved agents and can be adjusted by a person skilled in the art according to the condition of the subject, the type of condition(s) being treated, and the amount of a compound or composition being used. In certain embodiments, the topical compositions as described herein and the additional therapeutic agent are each administered in an effective amount (i.e., each in an amount that would be therapeutically effective if administered alone). In other embodiments, the topical compositions as described herein and the additional therapeutic agent are each administered in an amount that alone does not provide a therapeutic effect (a sub-therapeutic dose). In yet other embodiments, the topical compositions as described herein can be administered in an effective amount, while the additional therapeutic agent is administered in a sub-therapeutic dose. In still other embodiments, the topical compositions as described herein can be administered in a sub-therapeutic dose, while the additional therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a person in need thereof. Co-administration encompasses administration of a topical composition as described herein and one or more additional therapeutic agents in an essentially simultaneous manner, such as in a single pharmaceutical composition, for example, having a fixed ratio of first and second amounts, or as discrete dosage forms. In addition, such co-administration also encompasses use of each compound in a sequential manner in either order. When co-administration involves the separate administration of a first amount of a topical composition as described herein and a second amount of an additional therapeutic agent, they are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life, and kinetic profile.

In one or more embodiments where a topical composition as described herein is administered in combination with an additional therapeutic agent, the additional therapeutic agent may be any therapeutic agent that provides a desired treatment outcome. In particular, the additional therapeutic agent may be selected from known therapeutic agents for the treatment a skin disorder, muscle pain or arthritic pain, including one or more symptoms thereof. Such therapeutic agents will be known to those skilled in art.

Where a topical composition as described herein is administered in combination with an additional therapeutic agent, the additional agent may be administered in any “effective amount” which provides the desired therapeutic activity, as described above. Suitable dosage amounts and dosing regimens of the additional therapeutic agent can be determined by the attending physician and may depend on the particular condition being treated, the severity of the condition as well as the general age, health, and weight of the subject. It will be appreciated that, unless otherwise specified, dose ranges as described herein provide guidance for the administration of provided pharmaceutical compositions to an adult. The amount to be administered to can be determined by a medical practitioner or person skilled in the art.

The topical compositions of the present invention may be contained in a kit. The kit may include, for example, the topical composition together with administration or dosage instructions. The kit may further an additional therapeutic agent, wherein the topical composition and the additional therapeutic agent packaged or formulated individually, or packaged or formulated in combination. Thus, the topical composition may be present in a first container, and the kit can optionally include one or more agents in a second container. The container or containers may be placed within a package, and the package can optionally include administration or dosage instructions. The kits may optionally comprise instructions describing a method of using the topical composition in one or more of the methods described herein (e.g., for treating a skin disorder, muscle pain or arthritic pain).

Those skilled in the art will be aware that the invention described herein is subject to variations and modifications other than those specifically described. It is to be understood that the invention described herein includes all such variations and modifications. The invention also includes all such steps, features, methods, compositions, and compounds referred to or indicated in this specification, individually or collectively, and any and all combinations of any two or more of said steps or features.

Certain embodiments of the disclosure will now be described with reference to the following examples which are intended for the purpose of illustration only and are not intended to limit the scope of the generality hereinbefore described.

### EXAMPLES

### Example 1

A topical cream was prepared by combining vitamin C cream, aqueous niacinamide (distilled water), hyaluronic acid and cannabidiol to provide a composition as shown in Table 1. The resulting mixture was combined using a mechanical stirrer on low speed for 30 μminutes to provide a smooth cream, which was transferred to a container. The resulting cream was applied to the skin of 20 patients and in all cases increased absorption was observed compared to corresponding compositions in the absence of hyaluronic acid.

TABLE 1
Topical composition

| Component | Weight | |
|---|---|---|
| Vitamin C cream | 1 | kg |
| (Water, Coconut Oil*, Glycerin, Cetearyl | | |
| Alcohol, Stearic Acid, Polysorbate 60, Vitamin | | |
| C Ester, Phenoxyethanol, Vitamin E, Aloe | | |
| Barbadensis Leaf Juice*, Green Clay Extract, | | |
| Blueberry Fruit Extract*, Raspberry Fruit | | |
| Extract*, Cranberry Fruit Extract*, White | | |
| Willow Bark Extract*, Roseship Seed Oil, | | |
| Neem Seed Oil*, Rosemary Leaf Extract*, | | |
| Alcohol*, Xanthan Gum, Tetrasodium | | |
| Glutamate Diacetate) | | |
| Niacinamide/aqueous (1:1) | 50 g:50 g | |
| Hyaluronic acid (2000 kDa) | 10 | g |
| Pure cannabidiol (CBD) | 10 | g |

*organic

1. A topical composition comprising a cannabinoid, an extracellular matrix component or an extracellular matrix fragment, and a pharmaceutically acceptable carrier, excipient, diluent, reagent, or combinations thereof.

2. The composition of claim 1, wherein the cannabinoid is present at a concentration of about 0.5% (w/w) to about 20% (w/w), relative to the total weight of the composition.

3. The composition of claim 1, wherein the cannabinoid is selected from the group consisting of cannabidiol (CBD), cannabigerol (CBG), cannabinol (CBN), cannabichromene (CBC), Δ9-tetrahydrocannabivarin (THCV), and cannabivarin (CBV), cannabidivarin (CBDV), cannabinodiol (CBND), cannabielsion (CBE), cannabicyclol (CBL), cannabitriol (CBT), cannabidolic acid (CBDA), and isomers, and analogs thereof.

4. The composition of claim 1, wherein the extracellular matrix component or the extracellular matrix fragment is present at a concentration of about 0.5% (w/w) to about 20% (w/w), relative to the total weight of the composition.

5. The composition of claim 1, wherein the extracellular matrix component or the extracellular matrix fragment is an extracellular matrix protein or a fragment thereof.

6. The composition of claim 5, wherein the extracellular matrix protein is selected from the group consisting of elastin, collagen, fibronectin, lectin, laminin, merosin, tenascin, vitronectin, fibrillin, and combinations thereof.

7. The composition of claim 1, wherein the extracellular matrix component or the extracellular matrix fragment is an extracellular matrix polysaccharide or a fragment thereof.

8. The composition of claim 7, wherein the extracellular matrix polysaccharide is selected from the group consisting of hyaluronic acid, heparin sulfate, chondroitin sulfate, keratan sulfate, and combinations thereof.

9. The composition of claim 1, wherein the extracellular matrix component or the extracellular matrix fragment is hyaluronic acid or a fragment thereof.

10. The composition of claim 9, wherein the hyaluronic acid or the fragment thereof has an average molecular weight of about 2,000 Da to about 60,000 Da.

11. The composition of claim 1, wherein the extracellular matrix component or the extracellular matrix fragment is selected from the group consisting of hyaluronic acid, chondroitin sulfate, keratan sulfate, elastin, collagen, fibronectin, lectin, laminin, merosin, tenascin, vitronectin, fibrillin, syndecans, fragments thereof, and combinations thereof.

12. The composition of claim 1, further comprising a bioenhancer.

13. The composition of claim 12, wherein the bioenhancer comprises a P-glycoprotein inhibitor selected from the group consisting of piperidine, quercetin, genistein, naringin, sinomenine, glycyrrhizin, nitrile glycosides cuminum cyminum, zingiver officinale, lysergol, allium sativum, aloe vera, and combinations thereof.

14. The composition of claim 1, further comprising a component selected from the group consisting of a vitamin, steroid, anti-inflammatory, antibiotic, antiviral compound, and combinations thereof.

15. The composition of claim 1, wherein the composition is in the form of a cream, gel, ointment, tonic, lotion, soap, shampoo, or combinations thereof.

16. A method of treating a skin disorder, muscle pain, or arthritic pain, comprising topically administering to a patient in need of treatment a composition comprising a cannabinoid, an extracellular matrix component or an extracellular matrix fragment, and a pharmaceutically acceptable carrier, excipient, diluent, reagent, or combinations thereof.

17. The method of claim 16, wherein the skin disorder is a dermatological disease.

18. The method of claim 17, wherein the dermatological disease is selected from the group consisting of eczema, psoriasis, dandruff, sunburn, dermatitis (contact, atopic and seborrheic), poison ivy and conditions caused by other plant materials containing urushiol or related molecules, type 1 and type 2 herpes, insect bites, anal itching, vaginal itching, acne, warts, acute or chronic dermatoses, viral skin diseases, fungal skin diseases, scabies, melanoma, pyoderma, cellulitis, keratinocyte carcinoma, decubitus ulcer, and alopecia areata.

19. The method of claim 16, wherein the skin disorder is an undesirable skin tone, appearance, texture and/or structure, fine lines and/or wrinkles, reduced skin elasticity and/or sagging, reduced collagen production, signs of aging, dry skin, skin roughness, and combinations thereof.

20. Use of a cannabinoid, an extracellular matrix component or an extracellular matrix fragment, and a pharmaceutically acceptable carrier, excipient, diluent, reagent, or combinations thereof, in the manufacture of a medicament for treating a skin disorder, muscle pain, or arthritic pain by topical administration.
